(19) United States
(12) Patent Application Publication
(10) Pub. No.: US 2024/0026439 A1
(43) Pub. Date: Jan. 25, 2024

(54) METHOD FOR ENZYMATIC DISSOCIATION OF HYBRIDIZED PROBES IN SITU

(71) Applicant: 10x Genomics, Inc., Pleasanton, CA (US)

(72) Inventor: Hiroshi SASAKI, Dublin, CA (US)

(73) Assignee: 10x Genomics, Inc., Pleasanton, CA (US)

(21) Appl. No.: 18/213,214

(22) Filed: Jun. 22, 2023

Related U.S. Application Data
(60) Provisional application No. 63/355,059, filed on Jun. 23, 2022.

(51) Int. Cl. C12Q 1/6869; C12Q 1/6844; C12Q 1/6841

(52) U.S. Cl. CPC C12Q 1/6869; C12Q 1/6846; C12Q 1/6841

(57) ABSTRACT

The present disclosure relates in some aspects to methods and compositions for analyzing biological samples involving active stripping of detectably labeled probes from hybridization complexes such as rolling circle amplification products (RCPs). In some embodiments, the present application provides a method wherein double-stranded hybridized complexes are dissociated by DNA helicases. In some embodiments, single-stranded binding proteins facilitate the dissociation and prevent hybridization of the dissociated strands.

### CROSS-REFERENCE TO RELATED APPLICATIONS

This application claims priority to U.S. Provisional Patent Application No. 63/355,059, filed Jun. 23, 2022, entitled “METHOD FOR ENZYMATIC DISSOCIATION OF HYBRIDIZED PROBES IN SITU,” which is herein incorporated by reference in its entirety for all purposes.

### FIELD

The present disclosure relates in some aspects to methods and compositions for dissociating probes for detecting a target nucleic acid at a location in a biological sample.

### BACKGROUND

Methods are available for analyzing nucleic acids in a biological sample in situ, such as a cell or a tissue. For instance, advances in single molecule fluorescent in situ hybridization (smFISH), rolling circle amplification, and related methods have enabled high resolution detection of nucleic acid analytes in biological samples. However, highly multiplexed detection of target nucleic acids in biological samples typically requires multiple cycles of imaging and removal of signals from previous imaging cycles. The available methods of probe stripping using formamide, DMSO, or high temperature and of signal removal are associated with a number of drawbacks, including inefficiency. Thus, there is a need for improved methods for analyzing biological samples. The present application addresses this and other needs.

### BRIEF SUMMARY

In some aspects, provided herein is a method for analyzing a biological sample, which can comprise: a) contacting the biological sample with a first probe comprising a first recognition sequence that hybridizes to a target nucleic acid in the biological sample, wherein the first recognition sequence is complementary to a target sequence of one or more target sequences in the target nucleic acid; b) detecting a first signal associated with the first probe at a location in the biological sample; c) contacting the biological sample with an enzyme that exhibits a helicase activity, wherein the enzyme dissociates the first probe from the target nucleic acid; d) after contacting the biological sample with the enzyme in c), contacting the biological sample with a second probe comprising a second recognition sequence that hybridizes to the target nucleic acid, wherein the second recognition sequence is complementary to a target sequence of the one or more target sequences in the target nucleic acid; and e) detecting a second signal associated with the second probe at the location in the biological sample. In some embodiments, the first and second recognition sequences are the same sequence.

Provided herein is method for stripping probes in a sample, comprising contacting the biological sample with a nucleic acid probe comprising a recognition sequence that hybridizes to a target nucleic acid in the sample, wherein the recognition sequence is complementary to a target sequence in the target nucleic acid; detecting a signal associated with the nucleic acid probe in the sample; and contacting the sample with an enzyme that exhibits a helicase activity, wherein the enzyme dissociates the nucleic acid probe from the target nucleic acid thereby removing the signal associated with the nucleic acid probe in the sample. In some cases, the nucleic acid probe does not comprise a moiety that quenches a signal (e.g., a quencher moiety). In some cases, the nucleic acid probe and the target nucleic acid does not comprise a moiety that quenches a signal (e.g., a quencher moiety). For example, the nucleic acid probe does not comprise a hairpin-loop conformation. In some examples, the ends of the nucleic acid probe are not conjugated to a reporter and a quencher molecule.

In any of the preceding embodiments, one or more target sequences can comprise a first target sequence and a second target sequence that at least partially overlaps with the first target sequence, and wherein the first recognition sequence can be complementary to the first target sequence and the second recognition sequence can be complementary to the second target sequence. In any of the preceding embodiments, the target nucleic acid can be a nucleic acid probe or product thereof, and the one or more target sequences can be one or more barcode sequences in the nucleic acid probe or product thereof. In some embodiments, the target nucleic acid is a nucleic acid probe that hybridizes to a nucleic acid molecule in the biological sample and the one or more target sequences are one or more barcode sequences in an overhang region of the nucleic acid probe (e.g., a region of the probe that does not hybridize to the nucleic acid molecule in the biological sample). In some embodiments, the nucleic acid molecule in the biological sample is an endogenous nucleic acid analyte, a product thereof, or is comprised by a labeling agent. In any of the preceding embodiments, when the enzyme dissociates the first probe from the target nucleic acid (e.g., in c)), the biological sample can be contacted with a single-stranded binding protein prior to, simultaneously with, and/or after contacting with the enzyme. In any of the preceding embodiments, the single-stranded binding protein can bind to the target nucleic acid and/or the first probe.

In any of the preceding embodiments, the enzyme can be Rep-X (super helicase) Tte UvrD, RecQ, or a homolog or variant thereof. In any of the preceding embodiments, the enzyme can be active at temperatures ranging from 25-82 degrees Celsius. In any of the preceding embodiments, the enzyme can be contacted with the biological sample in a buffer comprising ATP. In any of the preceding embodiments, the buffer can comprise between 0.1 mM and 10 mM ATP. In any of the preceding embodiments, the enzyme can dissociate the first probe from the target nucleic acid in an ATP-dependent reaction. In any of the preceding embodiments, the single stranded binding protein can facilitate the dissociation of the first probe. In any of the preceding embodiments, the single stranded binding protein can remain bound to the target nucleic acid and/or the first probe dissociated from the target nucleic acid. In any of the preceding embodiments, the method can comprise removing the first probe from the biological sample. In any of the preceding embodiments, the first signal may not be detected at the first location after the removing of the first probe from the biological sample after using the enzyme to dissociate the first probe from the target nucleic acid (e.g., in c).

In any of the preceding embodiments, the method can comprise removing the enzyme from the biological sample prior to contacting the biological sample with the second probe in d). In any of the preceding embodiments, the method can comprise dissociating the single stranded binding protein from the target nucleic acid. In any of the preceding embodiments, the method can comprise washing the biological sample prior to contacting the biological sample with the second probe (e.g., in d). In any of the preceding embodiments, the first probe can be covalently or non-covalently bound to a first detectable label for producing the first signal, and the second probe can be covalently or non-covalently bound to a second detectable label for producing the second signal, wherein the first and second detectable labels can be the same or different.

In any of the preceding embodiments, the first probe can be covalently bound to the first detectable label and the second probe can be covalently bound to the second detectable label, wherein the first and second detectable labels are different.

In any of the preceding embodiments, the first probe can comprise a first overhang region and the second probe can comprise a second overhang region, wherein the first overhang region and the second overhang region can be the same or different. In any of the preceding embodiments, the first overhang region can correspond to the first signal and the second overhang region can correspond to the second signal. In any of the preceding embodiments, the method can comprise contacting the biological sample with a first detectably labeled probe that hybridizes to the first overhang region and a second detectably labeled probe that hybridizes to the second overhang region. In any of the preceding embodiments, the method can comprise contacting the biological sample with a first intermediate probe that hybridizes to the first overhang and a second intermediate probe that hybridizes to the second overhang, and can contact the biological sample with a first detectably labeled probe that hybridizes to the first intermediate probe and a second detectably labeled probe that hybridizes to the second intermediate probe. In any of the preceding embodiments, the first and/or second detectably labeled probe can comprise an optically detectable moiety. In some embodiments, the optically detectable moiety is a fluorophore.

In any of the preceding embodiments, the first probe, second probe, detectably labeled probes and/or the intermediate probes individually can be between about 5 and about 50 nucleotides in length. In some embodiments, the first probe does not comprise a moiety that quenches the first signal. In some embodiments, the second probe does not comprise a moiety that quenches the second signal. In some embodiments, the first probe does not comprise a moiety that quenches the first signal and the second probe does not comprise a moiety that quenches the second signal. In some embodiments, the target nucleic acid, the first probe and the second probe do not comprise a moiety that quenches a signal. In some embodiments, the first probe and the second probe are not molecular beacons. In some embodiments, the first probe and the second probe do not comprise a self-complementary sequence capable of forming a stem-loop structure in solution. In some embodiments, enzymatic dissociation of the first probe and the second probe from the target nucleic acid do not allow a signal to be generated without hybridization of a subsequent probe (e.g., directly or indirectly a detectably labeled probe). In some embodiments, enzymatic dissociation of the first probe and the second probe from the target nucleic acid does not release a signal from being suppressed or quenched.

In any of the preceding embodiments, the method can comprise f) contacting the biological sample with an enzyme that exhibits a helicase activity after detecting the second signal, wherein the enzyme in f) dissociates the second probe from the target nucleic acid. In any of the preceding embodiments, the method can comprise contacting the biological sample with a third probe comprising a third recognition sequence that hybridizes to a target nucleic acid in the biological sample, wherein the third recognition sequence can be complementary to a target sequence of the one or more target sequences in the target nucleic acid. In any of the preceding embodiments, the one or more target sequences individually can be between about 5 and about nucleotides in length.

In any of the preceding embodiments, contacting the biological sample with the first probe can comprise contacting the sample with a plurality of first probes that hybridize to a plurality of target sequences in the target nucleic acid, wherein each probe of the plurality of first probes comprises a recognition sequence complementary to one of the plurality of target sequences in the target nucleic acid. In any of the preceding embodiments, the enzyme can dissociate the plurality of first probes from the target nucleic acid. In any of the preceding embodiments, contacting the biological sample with the second probe can comprise contacting the sample with a plurality of second probes that hybridize to the plurality of target sequences in the target nucleic acid, wherein each probe of the plurality of second probes comprises a recognition sequence complementary to one of the plurality of target sequences in the target nucleic acid. In any of the preceding embodiments, the plurality of target sequences in the target nucleic acid can comprise one or more partially overlapping target sequences. In any of the preceding embodiments, the plurality of second probes can comprise the same recognition sequences as the plurality of first probes.

In any of the preceding embodiments, the first signal can be associated with the plurality of first probes and/or the second signal can be associated with the plurality of second probes. In any of the preceding embodiments, the target nucleic acid in the biological sample can be a deoxyribonucleic acid (DNA) or ribonucleic acid (RNA). In any of the preceding embodiments, the target nucleic acid in the biological sample can be an endogenous nucleic acid analyte in the biological sample. In any of the preceding embodiments, the target nucleic acid can be a nucleic acid probe or probe set that hybridizes to a nucleic acid molecule in the biological sample. In any of the preceding embodiments, the target nucleic acid in the biological sample can be a rolling circle amplification (RCA) product of a circular or circularizable probe or probe set that hybridizes to a nucleic acid molecule in the biological sample. In any of the preceding embodiments, the one or more barcode sequences can correspond to an analyte in the biological sample. In any of the previous embodiments, the method can comprise imaging the biological sample to detect the first and/or second signal. In some embodiments, the enzymatic dissociation of the first and/or second probe from the target nucleic acid does not generate, activate, or derepress a signal that is detected in the method. In any of the previous embodiments, the first and second probes can be configured such that the enzymatic dissociation of the first and second probes from the target nucleic acid is not capable of generating, activating, or derepressing a detectable signal, such as a fluorescent signal. In any of the previous embodiments, the first probe can be configured such that the enzymatic dissociation of the first probe from the target nucleic acid is not capable of generating, activating, or derepressing a detectable signal, such as a fluorescent signal, associated with the second probe. In any of the previous embodiments, the second probe can be configured such that the enzymatic dissociation of the second probe from the target nucleic acid is not capable of generating, activating, or derepressing a detectable signal, such as a fluorescent signal, associated with a subsequent probe (e.g., a third probe) that hybridizes to the target nucleic acid.

In some aspects, provided herein is a method for analyzing a biological sample can comprise: a) contacting the biological sample with a first probe, wherein the biological sample comprises a rolling circle amplification (RCA) product and the first probe hybridizes to a barcode sequence in the RCA product at a location in the biological sample; b) detecting a first signal associated with the first probe at the location in the biological sample; c) contacting the biological sample with an enzyme that exhibits a helicase activity, wherein the enzyme dissociates the first probe from the RCA product; d) removing the first probe from the biological sample, thereby extinguishing or removing the first signal at the location; e) contacting the biological sample with a second probe, wherein the second probe hybridizes to the barcode sequence in the RCA product at the location; and f) detecting a second signal associated with the second probe at the location in the biological sample.

In some aspects, provided herein is a method for analyzing a biological sample can comprise: a) contacting the biological sample with two or more different first probes or probe sets (first probe or probe set A and first probe or probe set B), wherein the biological sample comprises two or more different target nucleic acids A and B, and wherein the first probe or probe set A hybridizes to the target nucleic acid A, and the first probe or probe set B hybridizes to the target nucleic acid B; b) detecting a first signal associated with the first probe or probe set A and a first signal associated with the first probe or probe set B at one or more locations in the biological sample; c) contacting the biological sample with an enzyme that exhibits helicase activity, wherein the enzyme dissociates the first probes or probe sets from the target nucleic acids; d) contacting the biological sample with two or more different second probes or probe sets (second probe or probe set A and second probe or probe set B), wherein the second probe or probe set A hybridizes to the target nucleic acid A, and the second probe or probe set B hybridizes to the target nucleic acid B; and e) detecting a second signal associated with the second probe or probe set A and a second signal associated with the second probe or probe set B at the one or more locations in the biological sample at the one or more locations in the biological sample.

In any of the preceding embodiments, the target nucleic acids A and B can be rolling circle amplification products. In all previous embodiments the biological sample can be a cell or tissue sample. In any of the preceding embodiments, the tissue sample can be an intact tissue sample or a non-homogenized tissue sample. In any of the preceding embodiments, the tissue sample can be a tissue section. In any of the preceding embodiments, the tissue sample can be a fixed tissue sample, a frozen tissue sample, or a fresh tissue sample.

In any of the previous embodiments, the biological sample can be embedded in a matrix. In any of the previous embodiments, the target nucleic acid molecule can be crosslinked to one or more other molecules in the biological sample and/or a matrix embedding the biological sample. In any of the previous embodiments, the signal associated with the first probe and/or second probe can be associated with a signal amplification product generated in situ in the biological sample or in a matrix embedding the biological sample.

In any of the preceding embodiments, the signal amplification in situ can comprise RCA of a circular or circularizable probe that directly or indirectly binds to an endogenous nucleic acid analyte; hybridization chain reaction (HCR) on a probe that directly or indirectly binds to an endogenous nucleic acid analyte; linear oligonucleotide hybridization chain reaction (LO-HCR) on a probe that directly or indirectly binds to an endogenous nucleic acid analyte; primer exchange reaction (PER) on a probe that directly or indirectly binds to an endogenous nucleic acid analyte; assembly of branched structures formed on a probe that directly or indirectly binds to an endogenous nucleic acid analyte; or any combination thereof.

In some aspects, provided herein is a kit for analyzing a biological sample can comprise: a first probe comprising (i) a recognition sequence that is complementary to a target sequence in a target nucleic acid and (ii) a detectable label or a sequence corresponding to a detectable label; (b) a second probe comprising (i) a recognition sequence that is complementary to the target sequence in the target nucleic acid and (ii) a detectable label or a sequence corresponding to a detectable label; wherein the first and second probe comprise the same recognition sequence and different detectable labels or sequences corresponding to different detectable labels; and (c) an enzyme that exhibits a helicase activity. In the kit of the previous embodiment, the target sequence can be a barcode. In any of the preceding embodiments, the kit can comprise: (a) a plurality of first probes that each comprise (i) a recognition sequence that is complementary to a target sequence of a plurality of target sequences in a target nucleic acid and (ii) a detectable label or a sequence corresponding to a detectable label; and (b) a plurality of second probes that each comprise (i) a recognition sequence that is complementary to a target sequence of the plurality of target sequences in the target nucleic acid and (ii) a detectable label or a sequence corresponding to a detectable label. In the kit of the previous embodiments the enzyme can be Rep-X (super helicase), Tte UvrD, RecQ, or a homolog or variant thereof. In any of the preceding embodiments, the kit can comprise a single-stranded binding protein.

### BRIEF DESCRIPTION OF THE DRAWINGS

The following drawings illustrate certain embodiments of the features and advantages of this disclosure. These embodiments are not intended to limit the scope of the appended claims in any manner.

FIG. 1 shows an exemplary first hybridized product comprising a first detectable probe hybridized to a first target sequence in a target nucleic acid. As shown in FIG. 1 (1) the signal (e.g., fluorescent signal) associated with the detectable label on the first detectable probe is detected. An enzyme with helicase activity (e.g., helicase) binds to the first hybridized product (FIG. 1 (2) and (3)) and facilitates dissociation of the first detectable probe from the first target sequence in the target nucleic acid (FIG. 1 (4)).

FIG. 2 illustrates an exemplary method of analyzing a target nucleic acid (e.g., a rolling circle amplification product) using a first probe hybridized to a detectably labeled probe, a second probe hybridized to a detectably labeled probe, a helicase enzyme, and single-stranded binding proteins (SSB).

FIG. 3 illustrates an exemplary method of analyzing a target nucleic acid (e.g., a rolling circle amplification product) using a first detectably labeled probe, a second detectably labeled probe, a helicase enzyme, and single-stranded binding proteins (SSB).

### DETAILED DESCRIPTION

The present disclosure provides approaches to actively remove oligonucleotides from hybridization complexes using enzymes that exhibit helicase activities (e.g., DNA helicases) for analyzing a target nucleic acid at a spatially localized position in a biological sample. In any of the embodiments herein, the enzymes with helicase activities dissociate probes from hybridization complexes (e.g., hybridization complexes between probes and target nucleic acids, such as rolling circle amplification products). In some aspects, an enzyme with helicase activity dissociates probes from a hybridization complex in the presence of ATPs. In some aspects, the present disclosure provides alternative stripping approaches to facilitate multiplexed analysis of analytes (e.g., increased numbers of cycles of sequential probe hybridization and stripping to analyze a target nucleic acid in or associated with an analyte).

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Sequential hybridization of detectably labeled probes and/or detection oligonucleotides and imaging cycles in highly multiplexed imaging assays can lead to accumulation of detectably labeled probes carried over from previous imaging cycles ultimately causing decoding errors. This becomes more problematic for highly multiplexed imaging, as many rounds of labeling cycles are required. Current approaches to strip off the probes from target nucleic acids (e.g., RNA, cDNA, or amplification products such as rolling circle amplification products (RCPs)) include denaturation techniques such as using formamide, DMSO, or high temperatures to weaken DNA hybridization. Additional methods of removing probes include toehold displacement techniques involving competitively displacing probes; however, they may suffer from issues including specificity, slow reaction times, complex designs, and need for optimization to achieve high efficiencies. Existing approaches to remove detectable labels, such as chemical and/or enzymatic cleavage methods, including using DTT to cleave s-s bonds, photocleavable nucleotides, UDG and EndoVIII (USER enzymes), or photobleaching techniques are simple and easy to perform, however, may face challenges including the inability to photobleach all the detectable labels in the sample and/or the inability to image the same barcode more than once.

Multiple cycles of labeling and imaging in highly multiplexed imaging assays can lead to accumulation of detectably labeled probes from previous imaging cycles. The accumulated detection oligonucleotides are challenging to remove and can cause decoding errors. It is often challenging to strip off all detectable probes and associated signals even with extremely stringent wash conditions. For instance, the present inventors found that after 15 rounds of multiplexed imaging and extreme stripping conditions, including 80% formamide, 0.1% Tween-20 preheated to 80° C., two washes, two washes of ten minutes each at 47° C., some signals (e.g., detectable spots) from previous rounds appear to be to be not strippable. While this challenge is observed for many genes, this behavior is particularly obvious while imaging large clusters of highly expressed genes such as the transmembrane proteolipid protein gene (P1p1). Without being bound by theory, the potential reasons for these challenges include stickiness of hydrophobic fluorescent dyes and trapping by local gel-like environment. Existing techniques are thus inefficient at stripping off all signals. Improved approaches to actively strip off detectable probes (e.g., fluorescent oligonucleotides) from probes and/or products (e.g., RCPs) are needed.

The present disclosure accomplishes efficient stripping (e.g., active unwinding) of probes from target nucleic acids by contacting the biological sample with an enzyme having helicase activity (e.g., a DNA helicase) that actively unwinds double-stranded nucleic acids (e.g., double stranded DNAs formed by probes hybridized to a target nucleic acid). In some aspects, the probes are dissociated from the target nucleic acids in an ATP-dependent manner. In some embodiments, the probes are detectably labeled probes, or comprise one or more overhang regions for hybridization of a detectably labeled probe. In some cases, stripping of the probes from the target nucleic acid removes the detectable label, thereby extinguishing or removing signals associated with the probes and allowing detection of signals from subsequently hybridized probes at the same spatially localized positions in the biological sample. Various probes, target nucleic acids, and hybridization complexes according to the present disclosure are described in further detail in Section II.A below.

Enzymes having helicase activities include, but are not limited to, DNA helicases such as Rep-X (or super helicase), Tte UvrD helicase, Escherichia coli RecQ, or any suitable DNA helicase. The Rep-X or super helicase is an engineered ultra-processive helicase. The enzyme can unwind fluorescently labeled oligonucleotides that are 18 nucleotides in length. The Rep-X may be easily prepared using a well-established protocol. The Tte UvrD helicase exhibits helicase activity in an ATP-dependent manner at a reaction temperature of 65° C. The Tte UvrD helicase does not have high processivity and can potentially work without single-stranded binding proteins. The E. coli RecQ has ATP dependent activity and works between 25-37° C. Suitable enzymes that have helicase activities are described in further detail in Section II.B below.

In some aspects, the method comprises contacting the biological sample with a single-stranded DNA binding protein (SSB). In some embodiments, the SSB protein binds to single-stranded probes and/or target nucleic acids and prevents the unwound strands (e.g., the probes and/or target nucleic acid) from rehybridizing. Exemplary SSBs are described in further detail in Section II.C below.

In some aspects, a method provided herein comprises contacting the biological sample with a first probe comprising a first recognition sequence that hybridizes to a target nucleic acid in the biological sample, detecting a first signal associated with the first probe at a location in the biological sample, and contacting the biological sample with an enzyme that exhibits a helicase activity, wherein the enzyme dissociates the first probe from the target nucleic acid. In some embodiments, after contacting the biological sample with the enzyme, the method comprises contacting the biological sample with a second probe comprising a second recognition sequence that hybridizes to the target nucleic acid, and detecting a second signal associated with the second probe at the location in the biological sample. Various aspects of signal detection and analysis are described in further detail in Section II.D below.

Also provided herein are compositions, kits, and systems for enzymatic dissociation of probes in situ in a biological sample, as described in Section III below. The methods, compositions, and kits are suitable for analyzing any nucleic acid analytes or non-nucleic acid analytes associated with a target nucleic acid (e.g., via a labeling agent comprising or associated with a nucleic acid molecule). Exemplary biological samples and analytes are described in further detail in Section IV below.

### II. Enzyme-Mediated Dissociation of Hybridized Complexes

In some aspects, provided herein is a method for analyzing a biological sample, comprising: a) contacting the biological sample with a first probe which hybridizes to a first target sequence in a target nucleic acid; b) detecting a first signal associated with the first probe at a location in the biological sample; c) contacting the biological sample with an enzyme that exhibits a helicase activity, wherein the enzyme dissociates the first probe from the target nucleic acid; d) contacting the biological sample with a second probe which hybridizes to a second target sequence in the target nucleic acid; and e) detecting a second signal associated with the second probe at the location in the biological sample. In some embodiments, the first and second target sequences can be identical. In some embodiments, the target nucleic acid can be a nucleic acid probe or product thereof, and the first and second target sequences can be barcode sequences in the nucleic acid probe or product thereof. In some embodiments, the second target sequence at least partially overlaps with the first target sequence.

In some aspects, provided herein is a method for analyzing a biological sample, comprising: a) contacting the biological sample with a first probe comprising a first recognition sequence that hybridizes to a target nucleic acid in the biological sample, wherein the first recognition sequence is complementary to a target sequence of one or more target sequences in the target nucleic acid; b) detecting a first signal associated with the first probe at a location in the biological sample; c) contacting the biological sample with an enzyme that exhibits a helicase activity, wherein the enzyme dissociates the first probe from the target nucleic acid; d) after contacting the biological sample with the enzyme in c), contacting the biological sample with a second probe comprising a second recognition sequence that hybridizes to the target nucleic acid, wherein the second recognition sequence is complementary to a target sequence of the one or more target sequences in the target nucleic acid; and e) detecting a second signal associated with the second probe at the location in the biological sample.

### A) Polynucleotides and Hybridization Complexes

In some aspects, the methods disclosed herein comprise contacting a biological sample sequentially with nucleic acid probes (e.g., first and second probes) that hybridize to a target nucleic acid in the biological sample. In some embodiments, a probe according to the present disclosure comprises a recognition sequence (e.g., a sequence that is complementary to the target sequence in the target nucleic acid) capable of hybridizing to a target sequence in the target nucleic acid. In some embodiments, a target sequence comprises a barcode sequence. In some instances, the target nucleic acid is an endogenous target nucleic acid analyte (e.g., an endogenous DNA or RNA). In some cases, the target nucleic acid is a product of an endogenous nucleic acid (e.g., a cDNA). In some instances, the target nucleic acid is in a probe or other labeling agent bound to an endogenous analyte or product thereof. In some aspects, the target nucleic acid product may be an amplification product of a probe (e.g., a rolling circle amplification (RCA) product (RCP) of a circular or circularizable probe or probe set). The RCP may be a product of a hybridization complex comprising a circularizable probe or probe set (e.g., a padlock probe) bound to a nucleic acid molecule (e.g., an endogenous analyte such as endogenous DNA or RNA). Further aspects of target sequences and target nucleic acids according to the present disclosure are described in Section IV.C.

Any of the nucleic acid probe(s) (e.g., first and/or second probes) herein may comprise any of a variety of entities that can hybridize to a target nucleic acid, typically by Watson-Crick base pairing, such as DNA, RNA, LNA, PNA, etc. The nucleic acid probe(s) typically contains a hybridization region (e.g., a recognition sequence) that is able to bind to at least a portion of a target nucleic acid or product thereof, in some embodiments specifically. The nucleic acid probe(s) may be able to bind to a specific target nucleic acid (e.g., an mRNA, or other nucleic acids as discussed herein).

In some embodiments, the nucleic acid probe(s) (e.g., first probe, second probe) are directly or indirectly linked to a detectable label (e.g., a fluorophore), thereby associating the nucleic acid probe(s) with a signal produced by the detectable label. In some embodiments, the nucleic acid probe(s) is detectably labeled. In some embodiments, the nucleic acid probe(s) is detectably labeled prior to contacting with the biological sample. The nucleic acid probe(s) may be covalently or non-covalently bound to a detectable label (e.g., fluorescent moiety) by any suitable method. In some embodiments, the nucleic acid probe(s) are associated with a signal using detectably labeled probes that directly or indirectly bind to the nucleic acid probe (e.g., by hybridizing to the nucleic acid probe or to one or more intermediate probes that hybridize to the nucleic acid probe). The same signal (or absence thereof) can be reused in a signal code sequence. Thus, the first probe, second probe, and one or more subsequent probes can be associated with the same or different signals as part of the signal code sequence.

In some embodiments, one or more of the probes used in a method of analyzing a biological sample are not associated with a signal, and/or are associated with the absence of a signal. In methods comprising sequential hybridization of probes to decode a target sequence (e.g., barcode sequence), one or more dark cycles can be incorporated into the signal code sequence corresponding to the target sequence (e.g., barcode sequence) by contacting the sample with a probe (e.g., first and/or second probe) that is not detectably labeled and is not associated with a signal. In some embodiments, the probe does not comprise a sequence complementary to a detectably labeled probe (e.g., a detectably labeled probe in a universal pool of detectably labeled probes that is contacted with the biological sample). In some embodiments, the probe does not comprise an overhang region for hybridization of a detectably labeled probe. In some aspects, the absence of a detectable signal may be used as a “color,” e.g., in addition to the limited number of available fluorescent color channels in fluorescent microscopy, dark cycles can be used to alleviate issues associated with optical crowding in one or more color channels. Different probes may be detected, or distinguished from one another, by different labels, or by absence of a detectable label.

In some embodiments, the first and/or second probe is a detectably labeled probe. In some embodiments, a detectably labeled probe comprises a detectable moiety. In some embodiments, a detectably labeled probe comprises two or more detectable moieties. In some embodiments, a detectably labeled probe has one detectable moiety. In some embodiments, the detectable moiety is an optically detectable moiety (e.g., a fluorophore).

In some embodiments, the probes described herein (e.g., first probe, second probe, detectably labeled probes, and/or intermediate probes) independently are about 5, about 10, about 15, about 20, about 25, about 30, about 35, about 40, about 50 nucleotides in length.

In some embodiments, the methods disclosed herein comprise sequentially contacting a biological sample with a first probe and a second probe. In some embodiments, the first and second probes hybridize to first and second target sequences (e.g., first and second barcode sequences) in a target nucleic acid. In some embodiments, the first probe comprises a first recognition sequence that is complementary to the first target sequence in the target nucleic acid. In some embodiments, the second probe comprises a second recognition sequence that is complementary to the second target sequence in the target nucleic acid. In some embodiments, the first and second target sequences are identical (e.g., the first and second probe comprise the same recognition sequence). In some instances, the first and second probe comprise different recognition sequences. In some embodiments, the first and second target sequences at least partially overlap. In some aspects, the partially overlapping first and second target sequences are partially overlapping barcode sequences, e.g., in an RCP. In some embodiments, the methods disclosed herein comprise sequentially contacting the biological sample with any number of subsequent probes, which can be the same or different from the first and/or second probes. In some cases, the method comprises contacting the biological sample with a third probe comprising a recognition sequence (e.g., a third recognition region that is complementary to a target sequence of the one or more target sequences in the target nucleic acid) that hybridizes to the target nucleic acid. In some embodiments, the method comprises sequentially contacting the biological sample with up to 4, 5, 6, 7, 8, 9, 10, 11, 12, 13, 14, 15, 16, 17, 18, 19, 20, 21, 22, 23, 24, 25, 30, 40, 45, 50, or more probes in sequential contacting steps, wherein a signal or absence thereof of the probe is detected at the location in the biological sample in each contacting step. In some embodiments, the method comprises assigning a signal code sequence to a target sequence (e.g., a barcode sequence) in a target nucleic acid, and sequentially contacting the biological sample with probes comprising recognition sequences complementary to the target sequence (e.g., a barcode sequence), wherein each of the probes is associated with a signal or absence thereof that provides a “signal code” as part of the signal code sequence, until sufficient signals have been detected to determine the signal code sequence.

In some aspects, the recognition sequence of a probe is at least 5, at least 10, at least 15, at least 20, at least 25, at least 30, at least 35, at least 40, at least 50 nucleotides in length. In some embodiments, the recognition sequence is no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., in some embodiments, the recognition sequence is between 5 and 10 nucleotides in length, between 8 and 15 nucleotides in length, etc. In some aspects, a recognition sequence comprises about 4, 5, 6, 7, 8, 9, 10, 11, 12, 13, 14, 15, 16, 17, 18, 19, 20, 21, 22, 23, 24, 25, 26, 27, 28, 29, 30, 40, or more than 40 nucleotides. In some embodiments, the first probe, second probe, and one or more subsequent probes comprise the same recognition sequence. In some embodiments, the first probe, second probe, and one or more subsequent probes comprise different recognition sequences (e.g., for hybridizing to partially overlapping target sequences in the target nucleic acid molecule).

In some embodiments, the method comprises contacting the biological sample with an enzyme that exhibits a helicase activity between one or more of the probe contacting steps and a subsequent contacting step, wherein the enzyme dissociates a hybridized probe from the target nucleic acid, prior to contacting the biological sample with a subsequent probe. In some embodiments, the method comprises contacting the biological sample with an enzyme that exhibits a helicase activity between at least 2, at least 3, at least 4, at least 5, at least 6, at least 10, or more of the contacting steps and a subsequent contacting step. In some embodiments, the method comprises contacting the biological sample with an enzyme that exhibits a helicase activity between each contacting step.

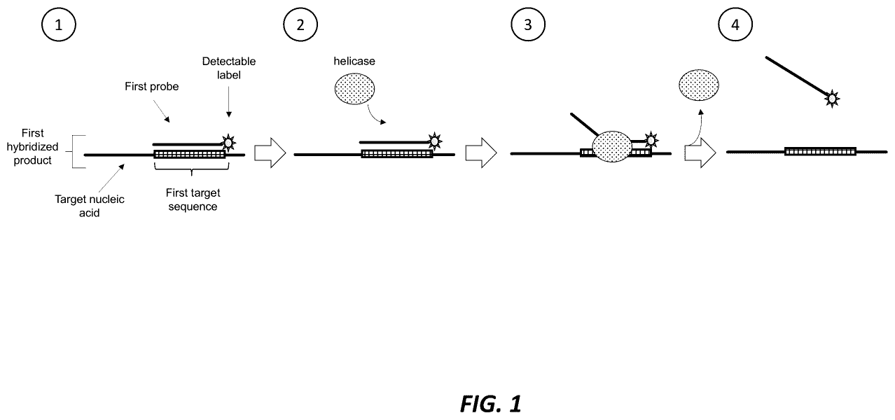

In some embodiments, the nucleic acid probe (e.g., first probes, second probes, and/or one or more subsequent probes described herein) is a linear probe (e.g., as shown in FIG. 1). In some embodiments, a nucleic acid probe (e.g., a first probe, second probe, and/or subsequent probe herein) comprises one or more overhang regions that do not hybridize to the target nucleic acid. In some embodiments, the one or more overhang regions comprises one or more sequences for hybridization of additional probes (e.g., detectably labeled probes or intermediate probes). In some embodiments, the overhang region comprises a barcode corresponding to a signal (e.g., a barcode corresponding to a particular detectable label). Thus, the probe (e.g., the first probe or second probe) is associated with the directly or indirectly binding a detectably labeled probe comprising said detectable label to the barcode corresponding to the detectable label.

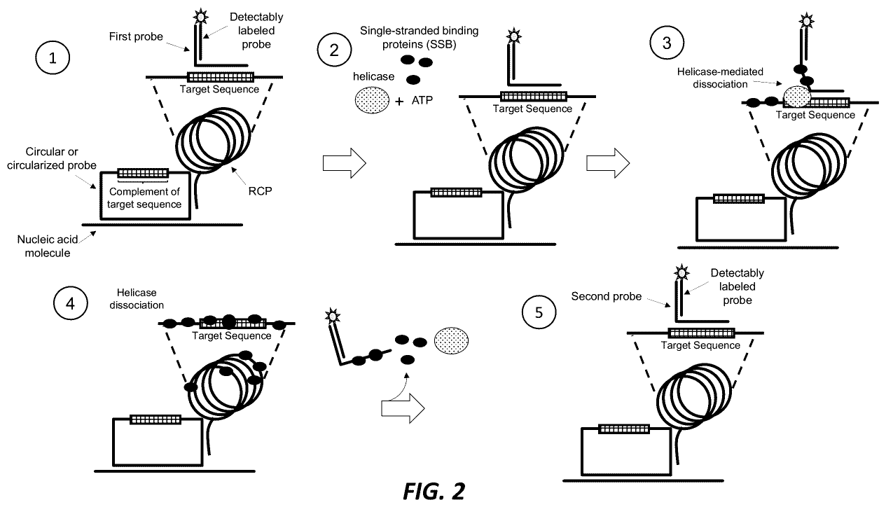

In some embodiments, the nucleic acid probes (e.g., first and/or second probes) are not directly conjugated to a detectable label. In some embodiments, the nucleic acid probes each comprise an overhang region that does not hybridize to the target nucleic acid (as shown in FIG. 2). In some aspects, the nucleic acid probes each comprise a region for hybridization with a detectably labeled probe. In some aspects, the nucleic acid probes each comprise one or more binding sites for an intermediate probe that directly or indirectly binds to a detectably labeled probe. In some aspects, the detectably labeled probe comprises a probe covalently or noncovalently bound to a detectable label.

In some embodiments, a probe herein (e.g., a first probe, second probe, and/or one or more subsequent probes) comprises one or more overhang regions (e.g., a region at the 5′ end and/or 3′ end of the probe that does not hybridize to the target nucleic acid). In some embodiments, the one or more overhang regions, individually, are between about 5 and about 50, between about 5 and about 40, between about 5 and about 30, between about 5 and about 20, between about 10 and about 50, between about 10 and about 45, between about 10 and about 35, between about 10 and about 30, between about 10 and about 25, between about 15 and about 50, between about 15 and about 40, or between about 15 and about 35 nucleotides in length. In some embodiments, the one or more overhang regions, individually, are at least 10, at least 15, at least 20, at least 25, at least 30, at least 35, at least 40, or at least 50 nucleotides in length. In some embodiments, one or more overhang regions, individually, are no more than 40, no more than 32, no more than 24, no more than 16, or no more than 8 nucleotides in length.

In some embodiments, the target nucleic acid sequence is an amplification product generated in situ using a probe that is circularized after binding a nucleic acid molecule in a sample. Exemplary circularizable probes or probe sets for generating rolling circle amplification products may be based on a circularizable probe or probe set (e.g., padlock probe), a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, etc. The specific probe or probe set design can vary. In some embodiments, a circular probe or a circularized probe is amplified through rolling circle amplification. In some embodiments, the circularizable probes or probe sets, such as a padlock probe or a probe set that comprises a padlock probe, contain one or more barcodes. In some embodiments, one or more barcodes are indicative of a sequence in the target nucleic acid, (e.g., a short sequence of about 5 nucleotides in length).

In some embodiments, the target nucleic acid is a rolling circle amplification (RCA) product of a circular or circularizable (e.g., padlock) probe or probe set that hybridizes to a DNA (e.g., a cDNA of an mRNA) or RNA (e.g., an mRNA) molecule in the biological sample. In some aspects, hybridization of an exogenous nucleic acid (e.g., primary probe such as a padlock probe) with an endogenous nucleic acid (e.g., mRNA) results in a rolling circle amplification product (RCP). In some aspects, the nucleic acid product is an amplification product (e.g., rolling circle amplification product (RCP)). In some embodiments, the RCA is linear RCA, a branched RCA, a dendritic RCA, or any combination thereof. In some aspects, the RCP is formed by the hybridization of a circularizable probe or probe set (e.g., a padlock probe). The circularizable probe or probe set (e.g., padlock probe) comprises at least two sequences that are complementary to sequences on an endogenous analyte (e.g., mRNA molecule). The circularizable probe or probe set may further comprise one or more barcode sequences that may enable identification of the circularizable probe or probe set bound to the analyte. The hybridization of the circularizable probe or probe set to the analyte followed by ligation and amplification may generate a rolling circle amplification product. The RCP generated by methods disclosed herein additionally comprise one or more target sequences (e.g., one or more target barcode sequences) that may be detected by contacting the sample with the first and second probe and an enzyme disclosed herein. The RCA reaction may be useful in multiplex situations or where sequential labelling is taking place (e.g., where multiple products are sequentially generated for each analyte), and/or in the analysis of the results, e.g., where the targets are detected by imaging. In some embodiments, the method further comprises a series of probe hybridization, detection, and probe dissociation steps to detect an analyte, optionally wherein the analyte is an RCA product. In some aspects, one or more of the probe dissociation steps comprises contacting the sample with an enzyme that exhibits a helicase activity.

In some embodiments, the target nucleic acid is a primary probe that hybridizes to a DNA (e.g., a cDNA of an mRNA) or RNA (e.g., an mRNA) molecule in the biological sample. In some aspects, hybridization of an exogenous nucleic acid (e.g., primary probe) with an endogenous nucleic acid (e.g., mRNA) results in a hybridization product that can be detected (e.g., as shown in FIG. 1). In some embodiments, the method comprises a series of probe hybridization, detection, and probe dissociation steps to detect a primary probe hybridized to the target nucleic acid. In some aspects, one or more of the probe dissociation steps comprises contacting the sample with an enzyme that exhibits a helicase activity.

A detectable moiety or label associated with any of the probes described herein can be a fluorophore, a radioisotope, a molecule capable of a colorimetric reaction, or any other suitable molecule or compound capable of detection. In some embodiments, the detectable label is a fluorescent moiety. In some embodiments, the detectable moiety is a chemical moiety for direct chemical, optical, or enzymatic detection. In some embodiments, the detectable moiety is a chemical moiety for direct chemical, optical, or enzymatic modification. In some embodiments, the detectable moiety is a chemical moiety for downstream chemical, optical, or enzymatic detection. In some embodiments, the detectable moiety is detectable by an optical (e.g., a dye or light) or a non-optical method. In some embodiments, a chemical moiety for chemical detection is biotin, DIG, or various other molecules that can be detected with a secondary binding molecule. In some embodiments, a chemical moiety for chemical detection is a di-thio linker. In some embodiments, a chemical moiety for optical detection is a fluorophore. In some embodiments, a chemical moiety for optical modification is biotin, DIG, or various other molecules that can be detected with a secondary binding molecule coupled to an enzyme (e.g., HRP) for signal amplification. In some embodiments, a chemical moiety for enzymatic modification is a modified nucleotide such as dUTP, which is cleavable using a combination of uracil-DNA glycosylase and AP endonuclease catalyzing enzymes. In some embodiments, the detectable moiety is joined or associated, directly or indirectly via a linker, to the probe.

In some embodiments, the probes (e.g., first and/or second probes) do not comprise a moiety that quenches a signal (e.g., a quencher moiety). A quencher can be an acceptor fluorophore that operates via energy transfer and re-emits the transferred energy as light. Other similar quenchers e.g., dark quenchers such as Dabsyl, Iowa Black, FQ, Iowa Black RQ, and others, do not re-emit transferred energy as light. Dark quenchers return to their ground states via nonradiative or dark decay when dissipated energy is given of via molecular vibrations such as heat. In some embodiments, dissociation of the probe from the target nucleic acid (e.g., using an enzyme that exhibits a helicase activity) does not result in quenching the signal associated with the probe.

In some embodiments, the first probe, second probe, subsequent probe, detectably labeled probes and/or the intermediate probes are between about 5 and about 50 nucleotides in length. In some aspects, the first probe, second probe, detectably labeled probes and/or the intermediate probes are between about 10 and about 20 nucleotides in length. In some aspects, the first probe, second probe, detectably labeled probes and/or the intermediate probes are between about 20 and about 30 nucleotides in length.

In some embodiments, the method comprises contacting the sample with a plurality of first probes comprising recognition sequences complementary to the plurality of target sequences in the target nucleic acid. In some embodiments, the plurality of first probes hybridize to partially overlapping target sequences in the target nucleic acid. In some embodiments, the plurality of first probes hybridize to non-overlapping target sequences in the target nucleic acid. In some embodiments, the plurality of first probes are designed to hybridize to 1, 2, 3, 4, 5, 6, 7, 8, 9, 10, 11, 12, 13, 14, 15 or more, 20 or more, 32 or more, 40 or more, or 50 or more target sequences tiling a target nucleic acid (e.g., an mRNA). In some embodiments, the plurality of first probes comprise different recognition sequences (e.g., to hybridize to the plurality of target sequences). In some embodiments, each probe of the plurality of first probes comprises a recognition sequence complementary to one of the plurality of target sequences in the target nucleic acid. In some cases, the plurality of first probes are associated with the same signal (e.g., comprise the same detectable label or the same sequence for hybridization of a detectably labeled probe in an overhang region of the first probe). In some embodiments, the method comprises contacting the sample with an enzyme that exhibits a helicase activity to dissociate the plurality of first probes from the target nucleic acid. In some embodiments, the method comprises contacting the sample with a plurality of second probes comprising recognition sequences complementary to the plurality of target sequences in the target nucleic acid. In some embodiments, each probe of the plurality of second probes comprises a recognition sequence complementary to one of the plurality of target sequences in the target nucleic acid. In some embodiments, the plurality of second probes hybridize to partially overlapping target sequences in the target nucleic acid. In some embodiments, the plurality of second probes hybridize to non-overlapping target sequences in the target nucleic acid. In some embodiments, the plurality of second probes are designed to hybridize to 1, 2, 3, 4, 5, 6, 7, 8, 9, 10, 11, 12, 13, 14, 15 or more, 20 or more, 32 or more, 40 or more, or 50 or more target sequences tiling a target nucleic acid (e.g., an mRNA). In some embodiments, the plurality of second probes comprise different recognition sequences (e.g., to hybridize to the plurality of target sequences). In some cases, the plurality of second probes is associated with the same second signal (e.g., comprise the same detectable label or the same sequence for hybridization of a detectably labeled probe in an overhang region of the first probe), wherein the second signal is the same or different from the signal associated with the plurality of first probes. In some embodiments, the plurality of second probes comprise the same recognition sequences as the plurality of first probes. The plurality of first probe and plurality of second probes are associated with the same or different signals. In some embodiments, the first signal is associated with the plurality of first probes and the second signal is associated with the plurality of second probes.

In some embodiments, the target sequence is a barcode sequence. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, the target nucleic acid comprises 1, 2, 3, 4, 5, 6, 7, 8, 9, 10, 11, 12, 13, 14, 15 or more, 20 or more, 32 or more, 40 or more, or 50 or more target sequences (e.g., barcode sequences). The barcode sequences may be positioned anywhere within the nucleic acid probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

A target sequence (e.g., a barcode sequence) may be of any length. In some embodiments, the one or more target sequences individually are between about 5 and about 50 nucleotides in length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of target nucleic acids or products thereof is less than the number of distinct targets (e.g., nucleic acid analytes and/or protein analytes) of the target nucleic acids or products thereof, and yet the distinct targets may still be uniquely identified from one another, e.g., by encoding a probe with a different combination of barcode sequences. However, not all possible combinations of a given set of barcode sequences need be used. For instance, each target nucleic acid or product thereof may contain 1, 2, 3, 4, 5, 6, 7, 8, 9, 10, 11, 12, 13, 14, 15, etc. or more barcode sequences. In some embodiments, a population of target nucleic acid probes or products thereof may each contain the same number of barcode sequences, although in other cases, there may be different numbers of barcode sequences present on the various target nucleic acids. In some embodiments, the barcode sequences or any subset thereof in the population of target nucleic acid probes or products thereof can be independently and/or combinatorially detected and/or decoded.

In any of the preceding embodiments, barcodes (e.g., primary and/or secondary barcode sequences) can be analyzed (e.g., detected) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of detectably labeled probes (e.g., detectably labeled first and/or second probes).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and WO2019199579A1, which are hereby incorporated by reference in their entirety.

In some aspects, the methods provided herein comprise contacting the sample with a library of first probes for analyzing multiple target sequences (e.g., target sequences in multiple different target nucleic acids) in the biological sample. In some embodiments, the probe (e.g., first probe) is comprised in a library of probes. In some embodiments, the library of probes further comprises another first probe that hybridizes to another target nucleic acid in the biological sample. In some embodiments, the method comprises contacting the biological sample with first probe library comprising two or more different first probes or probe sets (first probe or probe set A and first probe or probe set B), wherein the biological sample comprises two or more different target nucleic acids A and B, and wherein the first probe or probe set A hybridizes to the target nucleic acid A, and the first probe or probe set B hybridizes to the target nucleic acid B. In some embodiments, the method comprises detecting a first signal associated with the first probe or probe set A and a first signal associated with the first probe or probe set B at one or more locations in the biological sample. Next, the biological sample is contacted with an enzyme that exhibits helicase activity, wherein the enzyme dissociates the first probes or probe sets from the target nucleic acids. In some cases, the method can then comprise contacting the biological sample with a second probe library comprising two or more different second probes or probe sets (second probe or probe set A and second probe or probe set B), wherein the second probe or probe set A hybridizes to the target nucleic acid A, and the second probe or probe set B hybridizes to the target nucleic acid B; and detecting a second signal associated with the second probe or probe set A and a second signal associated with the second probe or probe set B at the one or more locations in the biological sample at the one or more locations in the biological sample. In some embodiments, a probe set is a plurality of probes that hybridize to the same target nucleic acid (e.g., probes that hybridize to target sequences tiling the target nucleic acid). In some embodiments, the target nucleic acids A and B are rolling circle amplification products.

In some embodiments, the library of first probes comprises first probes or probe sets that hybridize to 10 or more, 20 or more, 30 or more, 40 or more, 50 or more, 60 or more, 80 or more, 100 or more, 200 or more, 250 or more, 300 or more, or 1,000 or more different target nucleic acids in the sample (e.g., target nucleic acids in or associated with 10 or more, 20 or more, 30 or more, 40 or more, 50 or more, 60 or more, 80 or more, 100 or more, 200 or more, 250 or more, 300 or more, or 1,000 or more different endogenous analytes in the biological sample). In some embodiments, the library of second probes comprises second probes or probe sets that hybridize to 10 or more, 20 or more, 30 or more, 40 or more, 50 or more, 60 or more, 80 or more, 100 or more, 200 or more, 250 or more, 300 or more, or 1,000 or more different target nucleic acids in the sample (e.g., target nucleic acids in or associated with 10 or more, 20 or more, 30 or more, 40 or more, 50 or more, 60 or more, 80 or more, 100 or more, 200 or more, 250 or more, 300 or more, or 1,000 or more different endogenous analytes in the biological sample). Thus, in some embodiments, the method comprises detecting 10 or more, 20 or more, 30 or more, 40 or more, 50 or more, 60 or more, 80 or more, 100 or more, 200 or more, 250 or more, 300 or more, or 1,000 or more different target nucleic acids in the biological sample. In some embodiments, the methods provided herein are used for multiplex in situ hybridization or multiplex in situ sequencing or sequence detection.

In some embodiments, a library of first probes is contacted with a biological sample comprising a plurality of probes (e.g., primary probes) bound to nucleic acid molecules in the sample (e.g., endogenous nucleic acid analytes) or a plurality of products thereof (e.g., RCPs). In some embodiments, the biological sample is contacted with an enzyme that exhibits helicase activity. The enzyme dissociates the library of first probes from the plurality of probes (e.g., primary probes) or RCPs. In some embodiments, single-stranded binding proteins (SSBs) are contacted prior to, simultaneously with and/or after contacting with the enzyme. The SSBs may facilitate dissociation of the library of first probes from the probes (e.g., primary probes) or RCPs and may prevent rehybridization. In some embodiments, a library of second probes is contacted with the biological sample comprising the probes (e.g., primary probes) or RCPs. In some embodiments, the library of second probes may be detectably labeled. In some embodiments, the library of second probes may not be detectably labeled and may be contacted with a plurality of intermediate probes that may be directly or indirectly labeled with a detectable moiety (e.g., fluorescence moiety). In some aspects, the second detectable moiety is different from the detectable moiety of the library of first probes. In some aspects, the second detectable moiety is the same as the detectable moiety of the library of first probes.

In some aspects, the provided methods involve a step of contacting, or hybridizing, one or more polynucleotides, such as any of the probes described herein, to a cell or a sample containing a target nucleic acid with a region of interest in order to form a hybridization complex. In some aspects, the provided methods comprise one or more steps of ligating the polynucleotides (e.g., ligating two probes or the ends of one probe). In some instances, the method includes ligating the ends of a circularizable probe or probe set (e.g., a padlock probe) to form a circularized probe. In some aspects, the provided methods involve a step of amplifying one of the polynucleotides (e.g., a padlock probe or a circularized probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially.

In some embodiments, a method disclosed herein comprises contacting a biological sample with n sets of probes for n target sequences T1, . . . , Tk, . . . , Tn, in m cycles, wherein Probe Set 1 comprises P11, . . . , P1j, . . . , and P1m; Probe Set k comprises Pk1, . . . , Pkj, . . . , and Pkm; Probe Set n comprises Pn1, . . . , Pnj, . . . , and Pnm, and j, k, m, and n are integers, 2≤j≤m, and 2≤k≤n. In some embodiments, the biological sample is contacted with Probe Library P11, . . . , Pk1, . . . , and Pn1 in Cycle 1, with Probe Library P1j, . . . , Pkj, . . . , and Pnj in Cycle j, and Probe Library P1m, . . . , Pkm, . . . , and Pnm in Cycle m, and each probe comprises a sequence that hybridizes to a target sequence in T1, . . . , Tk, . . . , Tn, respectively. The probes may be detectably labeled or may bind to detectably labeled probes.

In some embodiments, in a particular cycle, the biological sample is contacted with a plurality of detection probes that hybridize to the probes contacted with the biological sample in the particular cycle. In some embodiments, the molecules comprising the n target sequences are rolling circle amplification (RCA) products. In some embodiments, T1, . . . , Tk, . . . , and Tn comprise barcode sequences B1, . . . Bk, . . . , and Bn, respectively, each corresponding to an analyte of interest, such as DNA, RNA, and/or protein molecules.

The present disclosure in some aspects provides a method for analyzing a biological sample, comprising: a) contacting the biological sample comprising a rolling circle amplification (RCA) product with a first detectable probe, wherein the first detectable probe hybridizes to a barcode sequence in the RCA product at a location in the biological sample; b) detecting a first signal or absence thereof associated with the first detectable probe at the location in the biological sample; c) contacting the biological sample with an enzyme that exhibits a helicase activity, wherein the enzyme dissociates the first detectable probe from the RCA product; d) removing the first detectable probe from the biological sample, thereby extinguishing or removing the first signal at the location; e) contacting the biological sample with a second detectable probe, wherein the second detectable probe hybridizes to the barcode sequence in the RCA product at the location; and f) detecting a second signal associated with the second probe at the location in the biological sample. In some embodiments, the method comprises dissociating the second probe or second signal associated with the second probe. In some cases, the second probe is dissociated by contacting the sample with an enzyme that exhibits a helicase activity. In some embodiments, the second probe is dissociated using conventional stripping methods (e.g., denaturation techniques such as using formamide, DMSO, or high temperatures to weaken DNA hybridization).

The present disclosure in some aspects provides a method for analyzing a biological sample, comprising: a) contacting the biological sample comprising a plurality of rolling circle amplification (RCA) products with a plurality of first probes, wherein the plurality of first probes hybridize to barcode sequences in the plurality of RCA products at one or more locations in the biological sample; b) detecting a plurality of first signals or absence thereof associated with the plurality of first probes at one or more locations in the biological sample; c) contacting the biological sample with a plurality of enzymes that exhibits helicase activity, wherein the enzyme dissociates the plurality of first probes from the plurality of RCA products; d) removing the plurality of first detectable probes from the biological sample, thereby extinguishing or removing the plurality of first signals at one or more locations; e) contacting the biological sample with a plurality of second probes, wherein the plurality of second probes hybridize to the barcode sequences in the plurality of RCA products at one or more locations; and f) detecting a second signals associated with the plurality of second probes at the one or more locations in the biological sample. In some cases, the plurality of second probes are dissociated by contacting the sample with an enzyme that exhibits a helicase activity. In some embodiments, the plurality of second probes are dissociated using conventional stripping methods (e.g., denaturation techniques such as using formamide, DMSO, or high temperatures to weaken DNA hybridization). In any of the embodiments herein, the steps of contacting the biological sample with a plurality of probes and detecting signals associated with the plurality of probes may be repeated with a plurality of third probes (e.g., comprising the same recognition sequences and associated with the same or different signals as the first and/or second plurality of probes).

In some embodiments, probes contacted with the biological sample in a particular cycle hybridize to their respective target sequences in the sample, which may then be contacted with detectably labeled probes (e.g., fluorescently labeled oligonucleotides) that hybridize to the probes. A probe in a particular cycle forms a probe complex with its corresponding detectably labeled probe and hybridizes to its target sequence. In some embodiments, the probe or probe complex is stripped from its target sequence in the next cycle by contacting the sample with helicase enzymes and single-stranded binding proteins. In some embodiments, a next set of probes are contacted in a subsequent cycle which hybridize to their respective target sequences in the sample. The next set of probes may be detectably labeled or may be bound to detectably labeled probes.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide. In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more barcodes of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some aspects, detection or determination of a sequence is performed such that the localization of a probe (e.g., primary probe) hybridized to the target nucleic acid in the originating sample is detected. In some embodiments, the assay comprises detecting the presence or absence of an amplification product or a portion thereof (e.g., RCA product). In some embodiments, the present disclosure provides methods for high-throughput profiling of one or more barcodes in a large number of targets in situ, such as transcripts and/or DNA loci, for detecting and/or quantifying nucleic acids in cells, tissues, organs or organisms.

### B) Helicases

Provided herein is a method for analyzing a biological sample, comprising contacting the biological sample with an enzyme that exhibits a helicase activity. In some embodiments, the enzyme dissociates a first probe (e.g., detectable probe) from a target nucleic acid (e.g., an endogenous nucleic acid such as an endogenous DNA or RNA, or a rolling circle amplification product of a probe hybridized to the endogenous nucleic acid). In some aspects, the helicase activity comprises unwinding of probes from hybridization complexes. In some embodiments, the helicase enzymes described herein do not have target specificity. In some embodiments, the helicase targets any double-stranded nucleic acid comprising open 3′ or 5′ ends. For instance, the helicase enzyme binds to a double-stranded complex, such as a complex formed by i) a detectably labeled probe (e.g., fluorescently labeled oligonucleotide) bound to ii) a target nucleic acid such as a rolling circle amplification product (as shown in FIG. 2). In some embodiments, the helicase unwinds in the 3′ to 5′ direction. In some embodiments, the helicase unwinds in the 5′ to 3′ direction. In some cases, the enzyme dissociates the first probe or the detectably labeled probe from the target nucleic acid in an ATP-dependent reaction. In some aspects, the unwinding activity is not ATP-dependent. The active unwinding activity of the helicase enzyme results in stripping of the detectably labeled probe from the target nucleic acid. In some embodiments, the enzymatic dissociation of the first probe and/or second probe from the target nucleic acid does not generate, activate, or derepress a signal that is detected in the method.

In some embodiments, the enzyme is active at temperatures ranging from 20-82° C. In some embodiments, the enzyme is active at temperatures ranging from 25-82° C. In some embodiments, the incubation with the enzyme that exhibits a helicase activity (e.g., a DNA helicase) is performed at 25-70° C., e.g., at 37° C. In some aspects, the incubation with the incubation with the enzyme that exhibits a helicase activity (e.g., a DNA helicase) is performed at 45-70° C. In some aspects, the incubation with the helicase is performed at 25° C. to 37° C. In some embodiments, the enzyme that exhibits a helicase activity is contacted with the biological sample in a buffer comprising ATP. In some embodiments, the buffer comprises between 0.1 mM and 10 mM ATP. In some embodiments, after contacting the biological sample with the enzyme that exhibits a helicase activity in a buffer comprising ATP and incubating the biological sample with the enzyme, the method comprises washing the biological sample and/or removing the ATP from the biological sample. An ATP removal step and/or a wash step using a wash buffer can remove the ATP by destabilizing the helicase bound to the DNA complex.

In some aspects, the method comprises incubating the sample with the helicase for at least 5 minutes, at least 10 minutes, at least 20 minutes, at least 30 minutes, at least 35 minutes, at least 40 minutes, at least 45 minutes, at least 50 minutes, or at least 60 minutes. In some embodiments, the incubation with the nuclease is performed for 20-60 minutes, 20-45 minutes, 30-120 minutes, 30-60 minutes, or 30-90 minutes. In some aspects, the method comprises incubating the sample with the helicase for no more than 50 minutes, no more than 60 minutes, no more than 70 minutes, no more than 80 minutes, or no more than 90 minutes.

Exemplary enzymes that exhibit helicase activities include, but are not limited tp, Rep-X (super helicase), Tte UvrD, RecQ, Rep, Rep-Y, RepD, PcrA-X, PcrA-DM or a homology (e.g., ortholog) or variant (e.g., mutant) thereof, and other helicase proteins that are substantially equal to helicases derived or isolated from Escherichia coli, e.g., proteins that are the same of similar in structure and/or function to the helicase protein of E. coli. Helicases belong to a class of proteins that are essential for DNA replication, transcription, translation, recombination, DNA repair, and ribosome biogenesis. Helicases are motor proteins that move directionally along a nucleic acid phosphodiester backbone, separating two hybridized nucleic acid strands using energy from ATP hydrolysis. In some embodiments, the helicase is an engineered DNA helicase.

In some embodiments, the enzyme that exhibits a helicase activity is an AddA-B, Rep-X super helicase or a homolog or variant thereof, multimers and/or crosslinked monomers of Rep-X, conformational variants of Rep-X, and Rep-X-like helicase proteins that are substantially equal to Rep-X derived or isolated from E. coli, e.g., proteins that are the same or similar in structure and/or function to the Rep-X protein of E. coli. Rep-X is an engineered E. coli Rep mutant in a closed conformation. Rep-X is an ultra-processive helicase that unwinds fluorescently-labeled oligonucleotides comprising at least 18 nucleotides. Rep-X can be easily engineered and has been demonstrated to have efficient activity in DNA-FISH applications.

In some embodiments, the enzyme that exhibits a helicase activity is Tte UvrD or a homolog or variant thereof, multimers and/or crosslinked monomers of Tte UvrD, conformational variants of Tte UvrD, and Tte UvrD-like helicase proteins that are substantially equal to Tte UvrD derived or isolated from the thermophilic organism Thermoanaerobacter tengcongensis, e.g., proteins that are the same or similar in structure and/or function to the Tte UvrD protein of T. tengcongensis. Tte UvrD Helicase is a repair helicase capable of unwinding double-stranded DNA in an ATP-dependent manner. Tte-UvrD helicase unwinds blunt-ended DNA duplexes as well as substrates possessing 3′- or 5′-ssDNA tails. In some cases, the substrate may have a double-stranded DNA fork at one end (e.g., a 3′-ssDNA tail comprising two overhangs or a 5′-ssDNA tail comprising two overhangs). Tte UvrD helicase is active on a wide range of DNA substrates. Tte UvrD helicase is thermostable and active at temperatures ranging from 45° C. to 70° C. The Tte UvrD helicase is not a high processive enzyme. In some aspects, the Tte UvrD helicase works without single-stranded binding proteins.

In some embodiments, the enzyme that exhibits a helicase activity is a RecQ helicase, RECQL, RECQ1, RECQL1, RECQ2, RECQ3, RECQ4, RECQ5, or a homolog or variant thereof, multimers and/or crosslinked monomers of RecQ, conformational variants of RecQ, and RecQ-like helicase proteins that are substantially equal to RecQ derived or isolated from E. coli, e.g., proteins that are the same or similar in structure and/or function to the RecQ protein of E. coli. RecQ DNA helicases are ubiquitous enzymes in bacteria and eukaryotes. RecQ proteins are ATP-dependent molecular motors that unwind double-stranded (ds) DNA. RecQ helicase is thermostable and active at temperatures ranging from 25° C. to 37° C.

In some aspects, method comprises contacting the biological sample with one type of helicase (e.g., 0.2-0.4 ng/μl of Tte UvrD helicase at 65° C.). In some aspects, the method comprises contacting the biological sample with one or more different types of helicases (e.g., two or more different helicases that work at the same temperature).

In some embodiments, the enzyme that exhibits a helicase activity is a DNA-DNA helicase. In some embodiments, the DNA-DNA helicase unwinds DNA-DNA hybrids but not DNA-RNA hybrids or RNA-RNA hybrids. In some embodiments, the target nucleic acid is a DNA molecule and the first probe is a DNA molecule, and the enzyme that exhibits a helicase activity is a DNA-DNA helicase.

In some embodiments, the enzyme that exhibits a helicase activity has DNA-RNA helicase activity and is capable of unwinding DNA-RNA hybrids. In some embodiments, the target nucleic acid is an RNA molecule (e.g., an mRNA), and the first probe comprises DNA. In some embodiments, the helicase dissociates one or more first probes comprising DNA from an RNA target nucleic acid in the biological sample. Exemplary enzymes exhibiting DNA-RNA helicase activity include Polθ-helicase, which efficiently unwinds RNA-DNA as described by Ozdemir, Ahmet et al. “Polymerase θ-helicase efficiently unwinds DNA and RNA-DNA hybrids.” The Journal of biological chemistry vol. 293, 14 (2018): 5259-5269, the content of which is herein incorporated by reference in its entirety. Additional examples of DNA-RNA helicases include but are not limited to DDX5 helicase, ATP-dependent RNA helicase A (DHX9), and senataxin (SETX).

Helicase proteins, including homologs and variants thereof, can be purified from an appropriate source, e.g., a prokaryotic (e.g., archaeal or bacterial) or a eukaryotic (e.g., fungal or mammalian) cell, by any suitable method. Alternatively, commercially available protein is also suitable for use in the methods described herein. Furthermore, synthetic protein can also be used. In some embodiments, the helicase is an engineered protein. In some embodiments, the proteins used to practice the methods described herein are not modified to include, or are not otherwise associated with, a fluorescent indicator, a chemiluminescent agent, an enzymatic label, a radioactive label, a ligand for a specific reporter molecule, e.g., biotin or digoxigenin, or any other related facilitator of detection, other than that the probes the proteins bind may include or be associated with such facilitator of detection.

Wild-type, homolog (e.g., ortholog), or variant (e.g., mutant) proteins can be used to practice the methods described herein. The following examples are intended to illustrate the breadth of sources where a protein may be derived or isolated from; in no aspect are they intended to be limiting. Non-limiting examples of Rep-X protein include Rep-X protein engineered from E. coli (Arslan S., et al., Science, 2015; 348(6232); 344-348), Tte UvrD protein include UvrD protein derived from T. tengcongensis (An L., et al., J Biol Chem, 2005; 280(32):28952-8), and RecQ protein include RecQ protein derived from E. coli (Bernstein D. A., et al., EMBO J, 2003: 22(19): 4910-4921).

### C) Single Stranded Binding Proteins

In some aspects, herein is a method for analyzing a biological sample, comprising a) contacting the biological sample with a first probe which hybridizes to a first target sequence in a target nucleic acid; b) detecting a first signal or absence thereof associated with the first probe at a location in the biological sample; c) contacting the biological sample with an enzyme that exhibits a helicase activity and with a single-stranded binding (SSB) protein, wherein the enzyme dissociates the first detectable probe from the target nucleic acid; d) contacting the biological sample with a second probe which hybridizes to a second target sequence in the target nucleic acid, and e) detecting a second signal associated with the second probe at the location in the biological sample. In some embodiments, the biological sample is contacted with the SSB protein prior to contacting the biological sample with the enzyme. In some embodiments, the biological sample is contacted with the SSB protein and the enzyme simultaneously (e.g., the SSB protein and the enzyme can be provided in the same composition). In some embodiments, the biological sample is contacted with the SSB protein after contacting the biological sample with the enzyme.

In some embodiments, the SSB protein binds to the target nucleic acid. In some instances, the SSB protein may prevent a stripped probe (e.g., first or second probes) from rehybridization to the target nucleic acid. In some embodiments, the SSB protein binds to single-stranded regions of the target nucleic acid. In some embodiments, the SSB protein binds to regions of the target nucleic acid that have been dissociated from the probe by the enzyme that exhibits a helicase activity. In some cases, the SSB prevents the target nucleic acid from rehybridizing to the dissociated probe. In some embodiments, the SSB facilitates the probe dissociation from the target nucleic acid by the helicase. In some cases, the target nucleic acid is a rolling circle amplification product. In some embodiments, the SSB binds to the first probe. In some embodiments, the SSB binds to single-stranded regions of the first probe (e.g., after dissociation of the first probe from the target nucleic acid). In some aspects, the SSB proteins bind to the target nucleic acid and/or the first probe as monomers. In some embodiments, the SSB proteins bind to the target nucleic acid and/or the first probe as multimers. In some embodiments, the binding to single-stranded DNA is in a sequence non-specific manner. In some aspects, the binding of the SSB to the target nucleic acid and/or the first probe facilitates the dissociation of the first probe from the target nucleic acid. The SSB protein can function with the helicase for the dissociation of a double-stranded DNA complex.

In some embodiments, once the target nucleic acids and detectable probes are dissociated from each other, the SSB protein can remain bound to the dissociated single stranded nucleic acids. In some embodiments, the SSB protein remains bound to the target nucleic acid and/or to the first probe dissociated from the target nucleic acid. In some embodiments, the first probe bound to the SSB is removed from the sample (e.g., in a wash step). In some embodiments, the SSB remains bound to the target nucleic acid in the biological sample after removal of the first probe. In some instances, a second probe bound to SSB proteins is contacted with the biological sample. A strand annealing protein (e.g., RecO) is then used to facilitate strand annealing of the two SSB-coated nucleic acids (e.g., SSB-coated target nucleic acid and SSB-coated second detectable probe). In some embodiments, the SSB proteins bind to the strand annealing protein to form a protein complex. In some embodiments, the protein complex is displaced from the duplex formed by the probe sequence and the target nucleic acid sequence. In some embodiments, the protein complex is kinetically blocked from binding to the duplex. In some embodiments, annealing between the minus strand and the plus strand is promoted by the binding of the strand annealing protein and the annealed strands are kinetically blocked from the protein complexes formed by the strand annealing protein and the first and/or second single-stranded binding proteins. The annealed duplex can then be detected, for instance, in situ in a sample, by detecting a signal associated with the second probe in the sample. In some embodiments, the strand annealing protein is selected from the group consisting of uvsY, RecO, RadB, and RAD52.

In some embodiments, the method further comprises dissociating the single-stranded binding protein from the target nucleic acid. This step is achieved by incorporating a wash step with a wash buffer. The washing step is performed to remove the SSB protein bound to the nucleic acids. The washing step may involve removing ATP from the biological sample thus destabilizing the SSB proteins.

In some embodiments, the SSB proteins are contacted with the biological sample to prevent hybridization (e.g., rehybridization) of the first probe to the target nucleic acid. In some aspects, single-stranded nucleic acids bound to SSB proteins do not hybridize or rehybridize in the absence of a protein (e.g., strand annealing protein such as RecO).

In some embodiments, the biological sample is not contacted with an SSB protein. Helicases such as Tte UvrD may function without the activity of SSB proteins. Alternative methods, including varying buffer conditions, for dissociating the double-stranded DNA complexes may also be used. In some aspects, formamide or DMSO is incorporated into the biological sample as an alternative to SSB proteins. Formamide and/or DMSO function by weakening DNA hybridization thereby dissociating the double-stranded DNA complexes. In some aspects, the sample may be subjected to high temperatures to facilitate dissociation of the double-stranded DNA complexes. Other alternatives, including but not limited to, alternative buffer conditions, chemical cleavage, enzymatic cleavage, photobleaching techniques, competitive displacement of labeled oligonucleotides with invading strands, may be used in conjunction with the helicase enzyme.

In some embodiments, the SSB is the SSB of E. coli or a variant thereof, the SSB of Mycobacterium tuberculosis or a variant thereof, the SSB of Deinococcus radiodurans or a variant thereof, the SSB of Thermus thermophiles or a variant thereof, the SSB from Sulfolobus solfataricus or a variant thereof, the human replication protein A 32 kDa subunit (RPA32) fragment or a variant thereof, the CDCl3 SSB from Saccharomyces cerevisiae or a variant thereof, the Primosomal replication protein N (PriB) from E. coli or a variant thereof, the PriB from Arabidopsis thaliana or a variant thereof, the SSB from T4 or a variant thereof, the SSB from RB69 or a variant thereof, or the SSB from T7 or a variant thereof. SSBs can be classified according to their sequence homology. The Pfam family, PF00436, includes proteins that all show sequence similarity to known SSBs. This group of SSBs can then be further classified according to the Structural Classification of Proteins (SCOP). SSBs fall into the following lineage: Class; All beta proteins, Fold; OB-fold, Superfamily: Nucleic acid-binding proteins, Family; Single strand DNA-binding domain, SSB. Within this family SSBs can be classified according to subfamilies, with several type species often characterized within each subfamily.

The single-stranded binding proteins, including variants thereof, can be purified from an appropriate source, e.g., a eukaryote (e.g., humans, mice, rats, fungi, protozoa, plants) or a prokaryote (e.g., bacteria, archaea, virus). In some aspects, the SSB proteins bind to the target nucleic acid and/or the first probe with high affinity as monomers (e.g., RPA70 of Saccharomyces cerevisiae) or multimers of different size units (e.g., hetero-tetramers of E. coli, Mycobacterium smegmatis and Helicobacter pylori or homo-dimers of Deinococcus radiodurans and Thermotoga maritima). The single-strand binding protein can be derived from the SSB of E. coli, the SSB of Mycobacterium tuberculosis, the SSB of D. radiodurans, the SSB of Thermus thermophiles, the SSB from Sulfolobus solfataricus, the human replication protein A 32 kDa subunit (RPA32) fragment, the CDCl3 SSB from S. cerevisiae, the Primosomal replication protein N (PriB) from E. coli, the PriB from Arabidopsis thaliana, the hypothetical protein At4g28440, the SSB from T4, the SSB from RB69, and the SSB from T7 or a variant thereof.

### D) Probe Detection and Analysis

In some aspects, after formation of a hybridization complex comprising nucleic acid probes described in Section II.A, the method further includes detection of the nucleic acid probes (e.g., first and/or second probes) hybridized to the target nucleic acid or any products generated therefrom or derivatives thereof. In some aspects, the provided methods are employed for in situ analysis of target nucleic acids, for example for in situ detection or multiplexed analysis in intact tissues or a sample with preserved cellular or tissue structure. In some aspects, the provided methods can be used to detect or determine the identity or amount in situ of target nucleic acids.

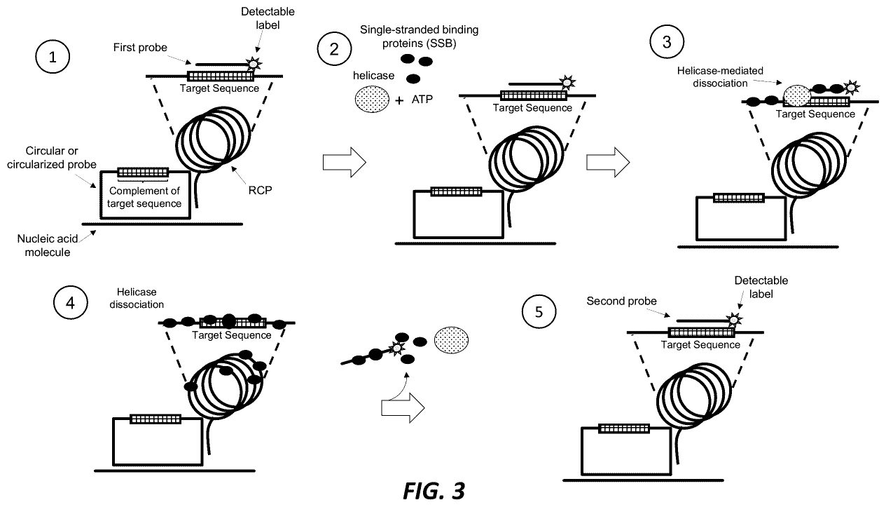

In some embodiments, the detecting step comprises contacting the biological sample with one or more probes (e.g., first and second probes) that directly or indirectly hybridize to a target nucleic acid or a rolling circle amplification product thereof. In some embodiments, the first probe is covalently or non-covalently bound to a first detectable label for producing the first signal, and the second probe is covalently or non-covalently bound to a second detectable label for producing the second signal. The first and second detectable labels may be the same or different. In some embodiments, the method comprises contacting the sample with a first probe (e.g., detectable probe or detectably labeled probe) which hybridizes to a first target sequence in a target nucleic acid or product thereof. In some embodiments, the step comprises detecting a first signal or absence thereof associated with the first probe (e.g., detectable probe or detectably labeled probe) at a location in the biological sample. For example, the first probe or second probe is detectably-labeled. In some cases, the first or second probe is a detectably-labeled probe that hybridizes to a primary probe as the target nucleic acid (e.g., as shown in FIG. 3). In some embodiments, the first probe comprises a first overhang region and the second probe comprises a second overhang region. In some instances, the first and second overhang regions may have the same sequences. For example, the overhang regions comprises a sequence not complementary to the target sequence. In some instances, the first and second overhang regions may have different sequences. In some embodiments, a first detectably labeled probe hybridizes to the first overhang region and a second detectably labeled probe hybridizes to the second overhang region (e.g., as shown in FIG. 2). In some embodiments, the first overhang region corresponds to the first signal and the second overhang region corresponds to the second signal. In some embodiments, the first or second probe is a detectably-labeled probe that hybridizes to an intermediate probe hybridized to the target nucleic acid. In some embodiments, a first intermediate probe hybridizes to the first overhang of a first probe and a second intermediate probe hybridizes to the second overhang of a second probe. A first detectably labeled probe may hybridize to the first intermediate probe and a second detectably labeled probe may hybridize to the second intermediate probe. The first signal may be detected using any suitable imaging technique described herein. In some embodiments, the step comprises contacting the biological sample with an enzyme (e.g., any of the helicase enzymes described herein), wherein the enzyme dissociates the first probe from the target nucleic acid. In some aspects, the biological sample is contacted with a single-stranded binding protein prior to, simultaneously with, and/or after contacting with the enzyme. The SSB proteins facilitate dissociation of the double-stranded DNA complexes (e.g., first probe bound to the target nucleic acid). In some embodiments, the step further comprises removing the first probe from the biological sample, thereby extinguishing or removing the first signal at the location. In some embodiments, a first signal is not detected at a first location after removing the first probe from the biological sample. The first probe may be removed from the biological sample using a wash step as described in Section II. The wash step may also be used to remove the ATP and destabilize the helicase and/or the SSB proteins bound to the probes. In some aspects, the helicase enzyme and the SSB proteins are removed from the sample prior to contacting the sample with a subsequent probe (e.g., second probe). Dissociation of the helicase and/or SSB proteins from the nucleic acids may be performed by any methods described herein. In some embodiments, the subsequent step comprises contacting the biological sample with a second probe (e.g., detectable probe or detectably labeled probe) which hybridizes to a second target sequence in the target nucleic acid. In some embodiments, the step comprises detecting the second signal associated with the second probe at the location in the biological sample. In some embodiments, the step further comprises contacting the biological sample with an enzyme that exhibits a helicase activity after detecting the second signal. In some embodiments, the enzyme dissociates the second probe from the target nucleic acid.

In some embodiments, the detecting, contacting with helicase and/or SSB proteins, dissociating, subsequent hybridizing and detecting steps are repeated with one or more detectably-labeled probes and/or one or more other detectably-labeled probes that directly or indirectly hybridize to the target nucleic acid or product thereof (e.g., RCP). In some aspects, a plurality of first and/or second probes are detectably labeled prior to contacting to the biological sample. The plurality of first probes sequentially hybridize to a plurality of target sequencing a plurality of target nucleic acids or products thereof. The plurality of first probes may be detectably labeled or may each comprise a hybridization region for a detectably labeled probe. The sample is imaged and a plurality of signals (e.g., first signals) are detected in situ. In some aspects, the plurality of first probes or detectably labeled probes may be dissociated from the plurality of target nucleic acids or products thereof by contacting with a plurality of helicases and SSB proteins. In some embodiments, a plurality of second probes is contacted with the biological sample for hybridization to a plurality of target nucleic acids. The sample is imaged and a plurality of signals (e.g., second signals) are detected in situ.

In some embodiments, provided herein are methods of detecting and analyzing one or more target sequences (e.g., barcode sequences) in target nucleic acids and/or in amplification products. In some embodiments, the method comprises a detecting signal (e.g., first or a second signal) associated with a probe (e.g., first or a second probe) in situ (e.g., at a location in the biological sample). The detecting step is followed by an active stripping step using a helicase enzyme. In some embodiments, the method comprises contacting the biological sample with a helicase enzyme to dissociate the probe (e.g., first or second probe) from the target nucleic acid. A subsequent wash step removes the probe from the biological sample thereby removing or extinguishing the signal at the location in the sample. In some embodiments, the detecting step precedes the helicase-assisted stripping step. Dissociation of the probe from the target nucleic does not produce a signal. In other words, hybridization of a detectable probe or a detectably labeled probe produces a signal, which is extinguished upon stripping of the probe from the target nucleic acid followed by removal of the probe from the sample. In some embodiments, the signal detection and active stripping steps are performed consecutively.

In some embodiments, provided herein are methods of detecting and analyzing one or more target sequences (e.g., barcode sequences) in target nucleic acids and/or in amplification products. In some embodiments, the method comprises detecting a signal (e.g., first or second signal) or absence thereof associated with a probe (e.g., first or second probe). One or more dark cycles can be incorporated into decoding the target sequences (e.g., barcode sequences) by contacting the sample with a probe (e.g., first and/or second probe) that is not detectably labeled. In some aspects, the absence of a detectable signal may be used as a “color,” e.g., in addition to the limited number of available fluorescent color channels in fluorescent microscopy, dark cycles can be used to alleviate issues associated with optical crowding in one or more color channels. In some embodiments, the signal code sequence includes one or more dark cycles as a “signal code” in the signal code sequence. Different probes may be detected, or distinguished from one another, by different labels, or by absence of a detectable label. In some embodiments, a probe may be directly or indirectly labeled with a detectable label which gives rise to a signal which may be recorded and/or assigned (e.g., serially) a signal code. In some embodiments, a probe is capable of hybridizing to a different target nucleic acid sequence (e.g., barcode sequence corresponding to a target analyte) and providing a signal. In some embodiments, a signal may include the signal detectable from the detectable label, and different detectable labels may provide different signals which may be distinguished, e.g., by color. In some embodiments, absence of signal may also be recorded and/or assigned a signal code. In some embodiments, in a plurality of probes, one or more of the probes may be lacking a detectable label, and thus the absence of a signal may be recorded and analyzed, for example, by assigning a signal code to the absence of signal (also known as a “dark” cycle for the one or more of the probes and the corresponding analyte(s)). In some embodiments, when there is a single cycle of detection to detect the signals from the probes, the plurality of probes (e.g., plurality of first and/or plurality of second probes) may comprise molecules of one probe which is associated with the absence of a signal, and the remainder of the probes may be associated with detectable labels which can be distinguished from one another (e.g., the probes may be detectably labeled with distinguishable labels, or may comprise distinct sequences for hybridizing to detectably labeled probes comprising distinguishable labels). In some embodiments, a combinatorial, e.g. sequential, labelling scheme is used (e.g., multiple cycles of sequential signal detection), and the plurality of probes (e.g., plurality of first and/or plurality of second probes) for different nucleic acid used in a given cycle need not all be distinguishable from one another in terms of the signal (e.g., may comprise the same detectable label, such as the same color of fluorophore), as it is the combination (e.g., sequence or order) of signals which identifies the target nucleic acid sequence, not a single signal.

In some embodiments, the detection may be spatial, e.g., in two or three dimensions. In some embodiments, the detection may be quantitative, e.g., the amount or concentration of a target nucleic acid may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc.

In some embodiments, provided herein are methods involving analyzing, e.g., detecting or determining, one or more target sequences (e.g., barcode sequences) in target nucleic acids and/or in amplification products, such as in an amplification product of a circularized padlock probe, which comprises one or more barcode sequences. In some embodiments, the analysis comprises determining the sequence of all or a portion of the amplification product. In some embodiments, the analysis comprises detecting a target sequence present in the amplification product. In some embodiments, the sequence of all or a portion of the amplification product is indicative of the identity of a region of interest in a target nucleic acid. In some embodiments, the analysis can be used to correlate a sequence detected in an amplification product to for instance, a circularizable probe or probe set (e.g., a padlock probe) (e.g., via a barcode). In some embodiments, the analysis can be used to correlate a sequence detected in the target nucleic acid to the probe used to generate the target nucleic acid (e.g., amplification product). In some embodiments, the detection of a sequence in an amplification product can provide information regarding the location and/or identity of an associated target nucleic acid (e.g., hybridized by the primary probe) in a sample. In some embodiments, due to amplification of one or more target nucleic acids (e.g., a padlock probe), particular sequences present in the amplification product or complementary sequences thereof can be detected even when a target nucleic acid is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region of interest in a target nucleic acid. In particular embodiments, the amplification product is an in situ rolling circle amplification (RCA) product of a circularized padlock probe.

In some aspects, the provided methods involve analyzing, e.g., detecting or determining, one or more sequences present in nucleic acid analytes (e.g., mRNA) and/or in a product or derivative thereof, such as in an amplified circular or circularizable probe. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a nucleic acid molecule to which the circular or circularizable probe hybridizes (e.g., a nucleic acid analyte or a nucleic acid molecule in a labeling agent as described in Section IV below).

In some aspects, a signal associated with the first probe and/or second probe is amplified in situ in the biological sample or in a matrix embedding the biological sample. In some embodiments, the signal amplification in situ comprises hybridization chain reaction (HCR) directly or indirectly on the first probe and/or second probe; linear oligonucleotide hybridization chain reaction (LO-HCR) directly or indirectly on the first probe and/or second probe; primer exchange reaction (PER) directly or indirectly on the first probe and/or second probe; assembly of branched structures directly or indirectly on the first probe and/or second probe; hybridization of a plurality of detectable probes directly or indirectly on the first probe and/or second probe, or any combination thereof.

In some embodiments, an amplification product can be detected using a first and second probe described herein. In some aspects, a signal associated with the first probe and/or second probe is or contributes to an amplified signal in situ in the biological sample or in a matrix embedding the biological sample. In some embodiments, the signal amplification in situ comprises RCA of a circular or circularizable probe that directly or indirectly binds to the analyte; hybridization chain reaction (HCR) directly or indirectly on a probe that directly or indirectly binds to the analyte; linear oligonucleotide hybridization chain reaction (LO-HCR) directly or indirectly on a probe that directly or indirectly binds to the analyte; primer exchange reaction (PER) directly or indirectly on a probe that directly or indirectly binds to the analyte; assembly of branched structures directly or indirectly on a probe that directly or indirectly binds to the analyte; or any combination thereof.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is incorporated herein by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is incorporated herein by reference in its entirety), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, WO 2020/102094, US 2022/0026433, WO 2020/163397, US 2022/0128565 and WO 2021/067475, US 2021/0222234 all of which are incorporated herein by reference in their entireties.

In some embodiments, hybridization chain reaction (HCR) can be used for signal amplification. A probe that directly or indirectly binds to the analyte can comprise an HCR initiator sequence or can comprise a hybridization sequence complementary to a hybridization region in an HCR initiator. In some embodiments, the method comprises contacting the sample with HCR monomers, wherein the probe comprises or is associated with an HCR initiator, and performing an HCR reaction on the probe. Thus, the probe is associated with an amplified signal by HCR. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an initiator nucleic acid molecule is introduced.

In some embodiments, similar to HCR reactions that use hairpin monomers, linear oligo hybridization chain reaction (LO-HCR) can also be detected with a signal associated with the first probe and/or a signal associated with the second probe. LO-HCR has been described in US2021/0198723, the content of which is incorporated herein by reference in its entirety.

In some embodiments, a signal associated with the first probe and/or a signal associated with the second probe is used to detect an amplification product generated using a primer exchange reaction (PER). In various embodiments, PER is performed using a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In some embodiments, the PER primer is comprised in or is associated with a probe that directly or indirectly binds to the analyte. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, hybridized probes or oligonucleotides or a product thereof may be contacted with a plurality of concatemer primers and a plurality of labeled probes. See for example, U.S. Pat. Pub. No. US20190106733, the content of which is incorporated herein by reference in its entirety, for exemplary molecules and PER reaction components.

In some embodiments, detection of nucleic acids sequences in situ includes an assembly for branched signal amplification. In some embodiments, the assembly complex comprises an amplifier hybridized directly or indirectly (via one or more oligonucleotides) to a sequence of a probe hybridized to an RNA substrate or a product thereof. In some embodiments, the amplifier is an intermediate probe comprising a hybridization region complementary to an overhang region of probe, and comprising multiple binding sites for probes (e.g., additional intermediate probes, first/second probes that are detectably labeled). In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), all of which are herein incorporated by reference in their entireties. In some embodiments, the enzyme that exhibits a helicase activity dissociates the branched hybridization complex.

In some embodiments, disclosed herein is a multiplexed assay where multiple targets (e.g., nucleic acids such as genes or RNA transcripts, or protein targets) are probed with multiple probes (e.g., padlock primary probes), hybridizing to the barcodes (or complementary sequences thereof) followed by sequential secondary barcode detection using multiple detectably labeled probes (e.g., first and/or second detectably labeled probes) and decoding of the detected signals. In some embodiments, detection of barcodes or subsequences of the barcode can occur in a cyclic manner.

In some embodiments, a method for analyzing a region of interest in a target nucleic acid is a multiplexed assay where multiple probes (e.g., circularizable probe or probe set such as padlock probes) are used to detect multiple regions of interest simultaneously (e.g., variations at the same location of a target nucleic acid and/or SNPs in various locations). In some embodiments, one or more detections of one or more regions of interest may occur simultaneously. In some embodiments, one or more detections of one or more regions of interest may occur sequentially. In some embodiments, multiple padlock probes of the same padlock probe design are used to detect one or more regions of interest, using different barcodes associated with each region of interest. In some embodiments, multiple padlock probes of different padlock probe design are used to detect one or more regions of interest, using different barcodes (e.g., each barcode associated with a target nucleic acid or sequence thereof). In some embodiments, the one or more regions of interest are localized on the same molecule (e.g., RNA or DNA). In alternative embodiments, the one or more single nucleotides of interest are localized on different molecules. The target nucleic acids or products thereof are then sequentially detected using probes, helicases and SSB proteins.

In some embodiments, each detectably labeled probe (e.g., detectably labeled first and/or second probe) in a set has a different target, e.g., a transcript or DNA locus. In some embodiments, two or more detectably labeled probes in a set have the same target. In some embodiments, two or more detectably labeled probes target the same transcript. In some embodiments, two or more detectably labeled probes target the same DNA locus. In some embodiments, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90 or 100 detectably labeled probes have the same target. In some embodiments, two or more detectably labeled probes target the same target or portion thereof. In some embodiments, five or more detectably labeled probes target the same target or portion thereof. In some embodiments, among other things, using multiple detectably labeled probes for the same target increases signal intensity. In some embodiments, each detectably labeled probe in a set targeting the same target interacts with a different portion of a target.

In some embodiments, all detectably labeled probes (e.g., detectably labeled first and/or second probes) for a target in a set have the same detectable moieties. In some embodiments, all detectably labeled probes are labeled in the same way. In some embodiments, all the detectably labeled probes for a target have the same fluorophore.

In some aspects, the provided methods comprise imaging an amplification product (e.g., RCP) and/or one or more portions of the target nucleic acids, for example, via binding of the detectably labeled probe (e.g., first and/or second probes) and detecting the detectable label. In some embodiments, the detectably labeled probe comprises a detectable label that can be measured and quantitated. In some embodiments, the detectable label is a directly or indirectly detectable moiety that is associated with (e.g., conjugated to) a molecule to be detected, e.g., a detectably labeled probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some cases, the detectable label is a fluorophore. In some embodiments, a fluorophore is a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Autofluorescence is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe (e.g., first or second probe) containing a detectable label can be used to detect one or more target nucleic acids and/or amplification products (e.g., RCP) described herein. In some embodiments, the methods involve incubating the detectable probe containing the detectable label with the sample and detecting the label, e.g., by imaging.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, 35S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. No. 4,757,141, US and U.S. Pat. No. 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, US U.S. Pat. No. 6,207,392, US 2002/0045045 and US 2003/0017264.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleic acid probe (e.g., detectably labeled first and/or second probe), and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, an antibody comprises an antibody molecule of any class, or any sub-fragment thereof, such as a Fab.

Other suitable labels for a nucleic acid probe (e.g., detectably labeled first and/or second probe) may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some aspects, the method comprises imaging the biological sample to detect the first and/or second signal. In some embodiments, the method comprises imaging the biological sample to detect a probe (e.g., first and/or second probe) hybridized to a target nucleic acid or product thereof. In some embodiments, a sequence of the ligation product, rolling circle amplification product, or other generated product is analyzed in situ in the biological sample. In some embodiments, the imaging comprises detecting a signal associated with a fluorescently labeled probe (e.g., detectably labeled first and/or second probes) that directly or indirectly binds to a target nucleic acid or product thereof (e.g., rolling circle amplification product of the circularized probe). In some embodiments, the sequence of the ligation product, rolling circle amplification product, or other generated product is analyzed by sequential hybridization and in situ detection.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectably labeled probe (e.g., detectably labeled first and/or second probe). In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the barcodes of the target nucleic acids or products thereof are analyzed using one or more decoding schemes to decode the signals, such as fluorescence, for sequence determination. In some embodiments, barcodes are analyzed (e.g., detected) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., first, second, third, fourth, . . . , nth probes). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; US B2; US 2016/0369329 A1; US 2021/0017587; and US 2017/0220733 A1, all of which are incorporated by reference in their entireties. In some embodiments, these assays enable signal amplification, combinatorial decoding, and error correction schemes at the same time.

In some aspects, the analysis and/or sequence determination can be carried out at room temperature for best preservation of tissue morphology with low background noise and error reduction. In some embodiments, the analysis and/or sequence determination comprises eliminating error accumulation as in situ detection proceeds.

In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

### III. Compositions, Kits, and Systems

In some embodiments, disclosed herein is a composition that comprises a complex containing a target nucleic acid, a probe, e.g., any of the probes described herein, an enzyme with helicase activity and/or single-stranded binding proteins.

In some embodiments, disclosed herein is a composition that comprises a target nucleic acid or product thereof bound to a detectable probe (e.g., first and/or second detectable probe or detectably labeled probe), wherein the hybridization complex is further bound to an enzyme with helicase activity and SSB proteins. In some aspects, an amplification product containing monomeric units of a target sequence complementary to a sequence of a target nucleic acid (e.g., mRNA molecule). In some embodiments, the amplification product is formed using any of the target nucleic acids, probes (e.g., circularizable probe or probe set such as padlock probes) and any of the amplification techniques described herein.

Also provided herein are kits, for example comprising one or more polynucleotides, e.g., any described in Section II.A, and reagents for performing the methods provided herein, for example reagents required for one or more steps comprising hybridization, ligation, amplification, detection, dissociation, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the probes are detectably labeled prior to contacting with the biological sample. In some embodiments, the kit further comprises a helicase, for instance for dissociating the detectable probe from the target nucleic acid or product thereof. In some embodiments, the helicase has unwinding activity. In some embodiments, the kit further comprises SSB proteins, for instance for facilitating dissociation and preventing rehybridization of the dissociated probes. In some embodiments, the kit further comprises a ligase, for instance for forming a ligated circular probe from the circularizable probe or probe set (e.g., padlock probe). In some embodiments, the ligase has DNA-splinted DNA ligase activity. In some embodiments, the kit further comprises a polymerase, for instance for performing amplification of the circularizable probe or probe set (e.g., padlock probe). In some embodiments, the polymerase is capable of using the ligated circular probe as a template for amplification. In some embodiments, the kit further comprises a primer for amplification.

Disclosed herein in some aspects is a kit for detecting a region of interest (e.g., barcode sequence) in a target nucleic acid, comprising: i) the target nucleic acid comprising a target sequence comprising a barcode sequence, ii) a detectable probe comprising a hybridization region complementary to the target sequence on the target nucleic acid or product thereof, and/or iii) a helicase enzyme, wherein: the hybridization regions in the target nucleic acid and the probe are capable of hybridizing to each other to generate a detectable signal. The helicase enzyme and/or SSB proteins facilitate dissociation of the hybridization complexes for hybridization with a subsequent detectable probe.

Disclosed herein in some aspects, is a kit for analyzing a biological sample comprising (a) a first probe comprising (i) a recognition sequence that is complementary to a target sequence in a target nucleic acid and (ii) a detectable label or a sequence corresponding to a detectable label; (b) a second probe comprising (i) a recognition sequence that is complementary to the target sequence in the target nucleic acid and (ii) a detectable label or a sequence corresponding to a detectable label. In some examples, the sequence corresponding to the detectable label is a sequence that is complementary to a detectably labeled probe. In some instances, the sequence corresponding to the detectable label is in a overhang region of the first or second probe (e.g., that does not hybridize to the target nucleic acid). In some embodiments, the first and second probe comprise the same recognition sequence and different detectable labels or sequences corresponding to different detectable labels (e.g., different overhang regions). In some embodiments, the kit comprises an enzyme that exhibits a helicase activity. In some embodiments, the target sequence is a barcode sequence. In some embodiments, the kit comprises (a) a plurality of first probes that each comprise (i) a recognition sequence that is complementary to a target sequence of a plurality of target sequences in a target nucleic acid and (ii) a detectable label or a sequence corresponding to a detectable label; and (b) a plurality of second probes that each comprise (i) a recognition sequence that is complementary to a target sequence of the plurality of target sequences in a target nucleic acid and (ii) a detectable label or a sequence corresponding to a detectable label. A sequence within a probe corresponding to a detectable label can hybridize to a detectably labeled probe (e.g., fluorescently labeled oligonucleotide). In some embodiments, the kit comprises an enzyme that exhibits a helicase activity. In some embodiments, the enzyme is Rep-X (super helicase), Tte UvrD, RecQ, or a homolog or variant thereof. In some embodiments, the kit comprises a single-stranded binding protein (SSB). In some embodiments, the kit comprises a buffer comprising ATP.

The various components of the kit may be present in separate containers or certain compatible components may be pre-combined into a single container. In some embodiments, the kits further contain instructions for using the components of the kit to practice the provided methods. In some embodiments, the proteins for dissociating hybridization complexes (e.g., helicases and/or SSB proteins) are provided separately from the plurality of probes. In some aspects, provided are instructions for coating the probes with the proteins for catalyzing dissociation.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection, such as barcode detection probes or detectable labels. In some embodiments, the kits optionally contain other components, for example nucleic acid primers, enzymes and reagents, buffers, nucleotides, modified nucleotides, reagents for additional assays.

### IV. Samples and Analytes

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, a cell pellet, a cell block, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface. In some embodiments, the biological sample is a tissue sample. In some embodiments, the tissue sample is an intact tissue sample or a non-homogenized tissue sample.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample. In some embodiments, the tissue sample is a fixed tissue sample, a frozen tissue sample, or a fresh tissue sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe or probe set (e.g., padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., padlock probe).

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for an analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable method of destaining or discoloring a biological sample may be used, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some embodiments, the target nucleic acid molecule is crosslinked to one or more other molecules in the biological sample and/or a matrix embedding the biological sample. In some aspects, the polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

(viii) Tissue Permeabilization and Treatment

In some embodiments, a biological sample can be permeabilized to facilitate transfer of species (such as probes) into the sample. If a sample is not permeabilized sufficiently, the amount of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Any suitable non-chemical permeabilization method may be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, one or more nucleic acid probes can be used to hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) to generate a product for analysis. In some aspects, when two or more analytes are analyzed, different probes that are specific for (e.g., specifically hybridize to) each RNA or cDNA analyte are used. For example, in some embodiments of the methods provided herein, templated ligation is used to detect gene expression in a biological sample. In some embodiments, gaps between the probe may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity or using any suitable method (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entireties.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a probe (e.g., first probe) that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed. Any of the products described herein can be detected using a method comprising contacting the biological sample with a first probe, detecting a first signal associated with the first probe at a location in the biological sample, contacting the biological sample with an enzyme that exhibits a helicase activity, wherein the enzyme dissociates the first probe from the target nucleic acid; contacting the biological sample with a second probe which hybridizes to a second target sequence in the target nucleic acid; and detecting a second signal associated with the second probe at the location in the biological sample.

(a) Hybridization

In some embodiments, the target nucleic acid is or comprises a hybridization product. In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. In some embodiments, the hybridization of an endogenous analyte to a primary probe can be detected. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets (e.g., primary probe, circularizable probe, or any probes described in Section II.A) can be hybridized to an endogenous analyte and/or a labelling agent and each probe may comprise one or more barcode sequences. Exemplary barcoded probes or probe sets may be based on a circularizable probe or probe set (e.g., padlock probe), a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

(b) Ligation

In some embodiments, the target nucleic acid is a ligation product. In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, a circularizable probe or probe set (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, the target nucleic acid is a primer extension or amplification product. In some embodiments, a product is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some embodiments, a primer extension reaction comprises any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Any suitable techniques for rolling circle amplification (RCA) may be used such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a moiety modification, a 5-Propargylamino-dCTP moiety modification, a N⁶-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, assays for the detection of numerous different analytes can use an RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a target sequence for a first and/or second probe as shown in FIGS. 1 and 2) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe. The exogenously added nucleic acid probe may be optionally ligated to a cellular nucleic acid molecule or another exogenous nucleic acid molecule. In other examples, a product comprising a target sequence for a probe disclosed herein (e.g., a target sequence for a first and/or second probe as shown in FIGS. 2 and 3) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a target sequence for a first and/or second probe as shown in FIG. 2) may be a probe hybridizing to an RCP. The probe may comprise an overhang that does not hybridize to the RCP but hybridizes to another probe. The probe may be optionally ligated to a cellular nucleic acid molecule or another probe, e.g., an anchor probe that hybridize to the RCP. In any of the embodiments herein, a plurality of cycles of hybridizing, detecting and then removing probes (e.g., fluorescently labeled probes) via enzymatic dissociation enable in situ detection of the bound probes to target nucleic acids, reporter oligonucleotides or generated amplification products associated with each reporter oligonucleotide.

C. Target Sequences

A target sequence for a probe disclosed herein (e.g., first probe or second probe) may be comprised in any analyte disclose herein, including an endogenous analyte (e.g., a viral or cellular nucleic acid, such as a target nucleic acid), a labelling agent, or a product of an endogenous analyte and/or a labelling agent.

In some embodiments, the target nucleic acid is an endogenous target nucleic acid analyte (e.g., an endogenous DNA or RNA). In some cases, the target nucleic acid is a product of an endogenous nucleic acid (e.g., a cDNA). In some cases, the target nucleic acid is a nucleic acid probe (e.g., primary probe that hybridizes to an endogenous nucleic acid). In some instances, the target nucleic acid is in a probe or other labeling agent bound to an endogenous analyte or product thereof. In some embodiments, the target nucleic acid is a nucleic acid probe or probe set that hybridizes to a nucleic acid molecule (e.g., endogenous DNA or RNA) in the biological sample. In some embodiments, the target nucleic acid product is an amplification product of a probe (e.g., a rolling circle amplification (RCA) product (RCP) of a circular or circularizable probe or probe set). As shown in FIGS. 1 and 2, the RCP may be a product of a hybridization complex comprising a primary probe such as circularizable probe or probe set (e.g., a padlock probe) bound to a nucleic acid molecule (e.g., an endogenous analyte such as endogenous DNA or RNA).

In some embodiments, the one or more target sequences comprise a first target sequence that at least partially overlaps with a second target sequence. In some aspects, the first target sequence and second target sequence are non-overlapping target sequences in the nucleic acid. In some embodiments, the first recognition sequence of a probe (e.g., first probe) is complementary to the first target sequence in the target nucleic acid. In some embodiments, the second recognition sequence of a probe (e.g., second probe) is complementary to the second target sequence in the target nucleic acid. In some aspects, the probes are designed to hybridize to 1, 2, 3, 4, 5, 6, 7, 8, 9, 10, 11, 12, 13, 14, 15 or more, 20 or more, 32 or more, 40 or more, or 50 or more target sequences tiling a target nucleic acid (e.g., an mRNA). In some embodiments, a plurality of first probes hybridize to a plurality of first target sequences in the target nucleic acid. In some examples, the first probes are designed to hybridize to 1, 2, 3, 4, 5, 6, 7, 8, 9, 10, 11, 12, 13, 14, 15 or more, 20 or more, 32 or more, 40 or more, or 50 or more target sequences tiling a target nucleic acid (e.g., an mRNA). In some embodiments, a plurality of second probes hybridize to a plurality of second target sequences in the target nucleic acid. In some cases, the first and second target sequences are the same. In some aspects, enzymatic dissociation can be used to remove a plurality of first probes hybridized to the plurality of first target sequences in the target nucleic acid.

In some embodiments, the one or more target sequences are one or more barcode sequences in the nucleic acid probe or product thereof. In some embodiments, the one or more barcode sequences correspond to an analyte (e.g., endogenous DNA or RNA) in the biological sample. Further aspects of barcodes according to the present disclosure as described in Section II.A.

### V. Applications

In some aspects, the provided embodiments can be applied in an in situ method of analyzing nucleic acid sequences, such as an in situ transcriptomic analysis, for example from intact tissues or samples. In some aspects, the embodiments can be applied in an imaging or detection method for multiplexed nucleic acid analysis. In some aspects, the provided embodiments can be used to identify or detect regions of interest in target nucleic acids.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tm for the specific sequence at a defined ionic strength and pH. The melting temperature Tm can be the temperature at which a population of double-stranded nucleic acid molecules becomes half dissociated into single strands. As indicated by standard references, a simple estimate of the Tm value may be calculated by the equation, Tm=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tm.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, SDS, 50° C. (also referred to as moderate stringency); and 3) low stringency: 1.0×SSPE, SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1× SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one probes, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following example is included for illustrative purposes only and is not intended to limit the scope of the present disclosure.

### Example 1: Helicase and Single-Stranded Binding Proteins (SSBs) Facilitate Dissociation Efficiency of Detectable Probes During In Situ Decoding

This example provides an exemplary workflow for a method of analyzing a biological sample comprising enzymatic dissociation of probes from a target nucleic acid. Multiple cycles of labeling and imaging in a highly multiplexed in situ assay can lead to accumulation of detectable probes from previous rounds of imaging. The accumulated detectable probes can be challenging to strip away, especially detectable probes that are bound to amplification products (e.g., RCPs). The example and the methods disclosed herein provide efficient stripping of detectable probes from nucleic acids or products thereof by using an enzyme that exhibits a helicase activity (e.g., a DNA helicase), optionally in combination with single-stranded binding proteins (SSBs).

A first probe comprising a recognition sequence complementary to a target sequence in a target nucleic acid (e.g., endogenous nucleic acid analyte) is introduced into a biological sample (e.g., a tissue sample) (FIG. 1). The first probe hybridizes to the complementary sequence such as the first target sequence in the target nucleic acid resulting in a first hybridized product. In an example, the first probe is a detectable probe comprising a detectable label (e.g., fluorescent label).

The signal associated with the first probe is detected by any suitable imaging techniques (e.g., fluorescent microscopy) at a location in the biological sample (FIG. 1 (1). Next, the sample is incubated with an enzyme that has helicase activity (e.g., DNA helicase such as Tte UvrD helicase) (FIG. 1 (2) and a suitable buffer supplemented with ATP at a temperature optimal for the helicase activity (e.g., 65° C. for Tte UvrD helicase). The helicase enzyme does not have target specificity and can bind to any double-stranded nucleic acid comprising open 3′ or 5′ ends. The helicase binds to the double-stranded first hybridized product consisting of the first probe and the first target sequence and having an open end (FIG. 1 (3)). The helicase exhibits either a 3′ to 5′ or 5′ to 3′ translocation directionality (e.g., Tte UvrD helicase exhibits a 3′ to 5′ translocation directionality) and unwinds the first probe from the first target nucleic acid in an ATP-dependent manner (FIG. 1 (4)). A wash buffer is used to wash off the dissociated first probe from the biological sample thereby removing the detectable label from the sample.

As shown in FIGS. 2 and 3, the target sequence is comprised in a rolling circle amplification product (RCP) formed by hybridization of a circular or circularizable probe to a nucleic acid molecule (e.g., cellular DNA or RNA). As shown in FIG. 2, the first probe comprises an overhang region (that does not hybridize to the target nucleic acid) comprising a sequence complementary to a detectably labeled probe of a pool of detectably labeled probes. In this case, the sample is contacted with a pool of detectably labeled probes that hybridize to the corresponding first probe(s). For example, the pool of detectably labeled probes can comprise four different detectably labeled probes labeled with distinguishable fluorophores, such as Cy3, Cy5, Cy7 and AF488. As shown in FIG. 3, the first probe is directly associated with a label (e.g., a detectable probe) and hybridizes to the target sequence in the RCP. The signal associated with the first probe is detected by any suitable imaging techniques (e.g., fluorescent microscopy) at a location in the biological sample.

Next, the sample is incubated with an enzyme that has helicase activity (e.g., DNA helicase such as Tte UvrD helicase) and a suitable buffer supplemented with ATP at a temperature optimal for the helicase activity (e.g., 65° C. for Tte UvrD helicase). The helicase enzyme does not have target specificity and can bind to any double-stranded nucleic acid comprising open 3′ or 5′ ends. The helicase binds to the double-stranded first hybridized product consisting of the first probe and the first target sequence and having an open end (FIG. 2 (3) and 3 (3)). The helicase exhibits either a 3′ to 5′ or 5′ to 3′ translocation directionality (e.g., Tte UvrD helicase exhibits a 3′ to 5′ translocation directionality) and unwinds the first probe from the first target nucleic acid in an ATP-dependent manner. In this example, the biological sample is contacted with a single stranded binding (SSB) protein and the helicase enzyme simultaneously (FIG. 2 (2) and 3 (2)). In some aspects, the biological sample is contacted with a single stranded binding (SSB) protein prior to or after contacting with the helicase enzyme. The SSB protein binds to the target nucleic acid and the first probe and may facilitate dissociation. The SSB protein prevents rehybridization of the first probe and the target sequence (FIG. 2 (4) and 3 (4)).

The biological sample is then contacted with a second probe comprising a recognition sequence complementary to the target sequence (FIG. 2 (5) and 3 (5)). The second probe is associated with a second signal that can be the same as the first signal or can be a different signal, as described above for the first probe. The sample is then imaged to detect the signal associated with the second probe at the location in the biological sample. Multiple sequential cycles of probe hybridization, imaging, and probe dissociation can be performed to analyze the target sequence (e.g., to determine a signal code sequence assigned to the target sequence).

It will be appreciated that the present example can be used for multiplexed analyte detection, wherein the sample is contacted with a plurality of first probes corresponding to a plurality of target nucleic acids, and a plurality of second probes corresponding to the plurality of target nucleic acids.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-58. (canceled)

59. A method for analyzing a biological sample, comprising:
contacting the biological sample with a helicase to remove a first probe comprising a first recognition sequence bound to a target nucleic acid in the biological sample, after detecting a first signal associated with the first probe at a location in the biological sample, wherein the first recognition sequence is complementary to a target sequence of one or more target sequences in the target nucleic acid and the helicase dissociates the first probe from the target nucleic acid;
after contacting the biological sample with the helicase, contacting the biological sample with a second probe comprising a second recognition sequence that binds to the target nucleic acid, wherein the second recognition sequence is complementary to a target sequence of the one or more target sequences in the target nucleic acid; and
detecting a second signal associated with the second probe at the location in the biological sample.

60. The method of claim 59, wherein the first and second recognition sequences are the same sequence.

61. The method of claim 59, wherein the one or more target sequences comprise a first target sequence and a second target sequence that at least partially overlaps with the first target sequence, and wherein the first recognition sequence is complementary to the first target sequence and the second recognition sequence is complementary to the second target sequence.

62. The method of claim 59, wherein the target nucleic acid is a nucleic acid probe or product thereof, and the one or more target sequences are one or more barcode sequences in the nucleic acid probe or product thereof.

63. The method of claim 59, wherein the biological sample is contacted with a single-stranded binding protein simultaneously with and/or after contacting with the helicase.

64. The method of claim 63, wherein the single-stranded binding protein binds to the target nucleic acid and/or the first probe.

65. The method of claim 59, wherein the helicase is Rep-X (super helicase), Tte UvrD, RecQ, or a homolog or variant thereof.

66. The method of claim 59, wherein the helicase is contacted with the biological sample in a buffer comprising ATP.

67. The method of claim 66, wherein the buffer comprises between 0.1 mM and 10 mM ATP.

68. The method of claim 59, wherein the helicase dissociates the first probe from the target nucleic acid in an ATP-dependent reaction.

69. The method of claim 63, wherein the single-stranded binding protein facilitates the dissociation of the first probe.

70. The method of claim 59, comprising removing the helicase from the biological sample prior to contacting the biological sample with the second probe.

71. The method of claim 59, wherein the first probe is covalently or non-covalently bound to a first detectable label for producing the first signal, and the second probe is covalently or non-covalently bound to a second detectable label for producing the second signal, wherein the first and second detectable labels are the same or different.

72. The method of claim 59, wherein
the first probe comprises a first overhang region and the second probe comprises a second overhang region, wherein the first overhang region and the second overhang region are the same or different; and the method comprises contacting the biological sample with a first detectably labeled probe that binds to the first overhang region and a second detectably labeled probe that binds to the second overhang region.

73. The method of claim 59, wherein the first and/or second probes do not comprise a moiety that quenches the first signal and the second signal, respectively.

74. The method of claim 59, wherein the method comprises contacting the biological sample with a helicase after detecting the second signal to dissociate the second probe from the target nucleic acid.

75. The method of claim 59, wherein the target nucleic acid in the biological sample is an endogenous nucleic acid analyte in the biological sample.

76. The method of claim 59, wherein the target nucleic acid in the biological sample is a rolling circle amplification (RCA) product of a circular or circularizable probe or probe set that binds to a nucleic acid molecule in the biological sample.

77. The method of claim 59, wherein the dissociation of the first and/or second probe from the target nucleic acid does not generate, activate, or derepress a signal.

78. The method of claim 59, wherein the biological sample is a cell or tissue sample.
